(19) United States
(12) Patent Application Publication
(10) Pub. No.: US 2014/0360403 A1
(43) Pub. Date: Dec. 11, 2014

(54) Organic-Inorganic Composite Materials as a Result of Biomineralization

(71) Applicant: Leibniz-Institut fuer Neue Materialien gemeinnuetzige GmbH, Saarbruecken (DE)

(72) Inventors: Ingrid Weiss, Saarbruecken (DE); Eva Weber, St. Ingbert (DE); Magdalena Eder, Heidenheim (DE); Eduard Arzt, Saarbruecken (DE); Andreas Simon Schneider, Saarbruecken (DE)

(21) Appl. No.: 14/369,325

(22) PCT Filed: Dec. 19, 2012
(86) PCT No.: PCT/EP2012/076198

§ 371 Date: Jun. 27, 2014

(30) Foreign Application Priority Data
Dec. 30, 2011 (DE) 10 2011 057 183.3

(51) Int. Cl. G01N 33/573

(52) U.S. Cl. CPC G01N 33/573
USPC 106/157.2; 536/23.2; 106/217.9; 435/7.4; 435/188; 435/258.1

(57) ABSTRACT

A method for producing organic-inorganic composite materials includes at least one biomineralising protein and/or polypeptide-encoding recombinant polynucleotide being introduced into at least one host cell, preferably from the class of slime moulds, and the expressed protein and/or polypeptide influencing the formation of inorganic particles in an extracellular matrix of the host cell.

The invention relates to a method of producing organic-inorganic composite materials by means of biotechnology and biomineralization, and the use thereof.

Biomineralization, in a very general sense, is the combination of inorganic solids by human beings, in particular under control of the structure, size and arrangement of said inorganic solids by an organic matrix. The resulting materials are also referred to as biominerals. Consequently, biominerals are composite materials composed of an inorganic component (an inorganic solid), generally of a few nanometers up to a few micrometers in size, and an organic component, usually an organic polymer. A particular property of these composite materials is their structure. For example, said composite materials often possess not only structuring at the level of the inorganic solids due to their form, size or morphology, but also higher-level structures, for example due to the arrangement of the inorganic solids along fibers or within layers. Such combinations of structures with different orders of magnitude are also referred to as hierarchic structures. These structures are often anisotropic. In this way, the structure or anisotropy of the organic material is reflected in the arrangement or structure of the inorganic solids.

The term biomineral does not mean that the inorganic component is always crystalline like a mineral but that it may also constitute amorphous or semi-crystalline solids.

The organic component comprises biopolymers which, in the broadest sense, are biologically occurring polymers and, for composite materials, are preferably stabilizing substances such as, for example, lignin, collagen, chitin or cellulose which are generated by supporting tissue cells.

Known examples of such organic-inorganic composite materials are skeletons of mussels, snails, sea urchins but also the bones of mammals, egg shells or the exoskeleton of arthropods. In many cases, combining an organic matrix with an inorganic component which often has a particular structure or form results in not only a surprising strength but also a high degree of elasticity which the pure inorganic or organic component would not have.

A recent review article (Weiss I. M. and Marin F. Met. Ions. Life Sci. 2008, 4, 71-136, The Role of Enzymes in Biomineralization Processes) gives an overview of the current state of research on the proteins and other organic substances involved in the composition of biominerals. Another publication (Weiss I. M., ChemBioChem 2010, 11, 297-300) describes the importance of the organic matrix and combination of various proteins for the selective spatially directional arrangement of aragonite. These references are hereby explicitly incorporated in the description.

For some composite materials, for example, some of the proteins contributing to the formation have already been isolated and characterized. These are often proteins which are capable of binding ions and in this way can promote the formation of solids from solutions, in particular also with control of the morphology of the inorganic solid.

WO 2007/125127 A2, for example, describes the selective production of aragonite by recombinantly obtained perlucin.

However, other processes may also influence the formation of the composite materials. For example, proteins may catalyze chemical reactions such as reductions, oxidations or other reactions which contribute to the formation of the inorganic structure. Examples of these are silicateins which catalyze the formation of polymers of silicic acid. Other proteins may increase the concentration of precursors needed for the formation of the inorganic solids. For example, carbonic anhydrases may provide hydrogencarbonate for the formation of carbonates by hydration of CO₂.

A biomineralizing protein may of course also include several domains with different functions for biomineralization and thus, for example, may have both possibilities of binding ions and reaction centers for chemical reactions. Thus, for example, nacrein, a protein which plays a role in the production of mother-of-pearl, includes two carbonic hydrase domains and a possible binding domain for calcium ions.

Aside from the binding of ions, the control of other parameters for specific control of biomineralization is also important. Thus, for example, the structure of the hydrate sheath at the binding site of the inorganic solid or the local concentration of ions also influences the structure of the resultant solid.

As a result, frequently a whole range of proteins is involved in producing the structures of nature, some of which are highly complex. It is also possible, however, that low amounts of individual proteins serve as crystallization nucleus for forming crystals.

As discussed above, incorporation of inorganic materials in organic matrices with the formation of an organic-inorganic composite material results in novel properties. Most biopolymers, however, have no inorganic components or only a low inorganic component content.

WO 98/36084 A2 discloses that incorporation of calcium-binding proteins may improve plant growth. This also involved introducing a hydroxyapatite-inducing enzyme into plant cells. However, the protein was expressed inside the plant. No biomineralization occurred.

It was an object of the invention to provide a method of producing novel organic-inorganic composite materials which can be prepared in a simple manner in self-producing systems. In particular, the method enables already known structures from biopolymers to be provided with inorganic components.

### Solution

This object is achieved by the inventions with the features of the independent claims. Advantageous embodiments of the inventions are characterized in the dependent claims. The wording of all claims is hereby incorporated by reference into the present description. The invention also encompasses any useful combinations and more specifically any combinations mentioned of independent and/or dependent claims.

Surprisingly, it has been found that the biomineralizing systems can be identified by a method in which at least one recombinant polynucleotide is introduced into at least one host cell, preferably from the class of slime molds (Eumycetozoa), said recombinant polynucleotide encoding at least one protein and/or polypeptide, and the first recombinant polynucleotide being suitable for expressing the encoded protein and/or polypeptide in the host cell. Said protein and/or polypeptide is expressed in the host cell. Subsequently, the expressed protein is investigated for a biomineralizing action. This means that the expressed protein and/or polypeptide influences, in particular stimulates or increases, the formation of inorganic particles in an extracellular matrix of the host cell.

This method enables proteins to be screened in a simple manner for their possible biomineralizing action. Thus it is possible, for example, to select a host cell which, for example, allows rapid screening of various proteins, owing to their rapid sequence of generations. After the proteins have been found, the sequences found may be transferred to the second host cell.

Thus, in the using the method, it is possible to test a large number of possibilities and variants of proteins, for example size of the proteins, arrangement of functional groups, mechanical flexibility and solvent compatibility of the basic structure and in combination with an organic matrix.

Consequently, the organic component of the organic-inorganic composite material consists of at least the extracellular matrix of the cell. Preference is given to the inorganic component present in the composite material being inorganic particles.

The invention inter alia also enables already known biopolymers to be provided with new properties. In this context, the method may utilize already known structures, more particularly the hierarchic structure of many biopolymers and the characteristic reaction thereof with aqueous solutions (this includes both solid and gel- or slime-like extracellular matrices consisting of evolutively optimized combinations of different biopolymers). By utilizing natural processes, the method is particularly energy-efficient. At the same time, by potentially employing efficient screening techniques, it allows rapid and specific adaptation of the properties of the organic-inorganic composite material.

Moreover, since the organic-inorganic composite materials are based on self-reproducible systems, they may already be duplicated and prepared.

“Biomineralizing” for the purposes of the application means generally the ability to bring about a controlled phase transition of an inorganic material. This may be the formation of an inorganic material from a solution but also a change in morphology or modification (including phase transitions, e.g. amorphous/crystalline) of an inorganic material. Preference is given to an inorganic material being formed. The formed material here may also be amorphous or semi-crystalline.

A protein and/or polypeptide with biomineralizing action for the purposes of the application means a protein and/or polypeptide which causes the generation of inorganic solids by binding ions, said binding also comprising promoting the formation of a crystallization nucleus, and/or by a chemical reaction, said chemical reaction also including electron transfer processes such as oxidation or reduction. A protein of this kind may therefore either have biomineralizing action itself or else modify or influence other substances, in particular proteins or polymers, in such a way that the latter develop biomineralizing action, for example by modifying the organic matrix or changing the local concentration of precursors for the inorganic particles. An example of such a modification may be glycosidation, phosphorylation, sulfation, hydroxylation, acetylation or deacetylation. Preference is given to proteins having per se biomineralizing action.

Biomineralizing action further comprises binding the protein or polypeptide to already present interfaces of inorganic solids whose morphology, modification or phase is changed as a result of said binding. Anti-freeze proteins which are present both in animals and in plants have been known to have comparable effects. In certain circumstances, this effect may result in the inorganic solid being dissolved or moved to other tissue parts of the organism.

“To culture” for the purposes of the present application means the propagation of the host cell, which also includes differentiation of the propagated host cells, for example into different tissues of a plant cell or differentiation into stem and fruiting body cells.

“To express” or “expression” means production of the protein and/or polypeptide encoded by a polynucleotide in the chosen host cell. This includes transcription and translation of the information on said polynucleotide. In addition, the protein and/or polypeptide formed may also be modified further by post-translational processes in the cell.

“Producing” the biomineralizing action means utilization of the biomineralizing action of the protein. This may be done on the one hand as part of expressing the protein in the cell or during incorporation into the cell wall. However, further treatments of the cell with precursors for a biomineral, for example solutions of ions, are also included. It may also be necessary to add these ions already during culturing of the host cell.

Individual steps of the method are described in more detail below. Said steps need not be carried out necessarily in the order indicated, and the method to be illustrated may also have other steps which are not mentioned.

In order to obtain the host cell with a polynucleotide of the invention, any techniques and methods known to the skilled worker may be used, depending on the type of cell. These include, for example, transforming, transfecting or transducing said cell with plasmids, phagemids, cosmids, retroviral or adenoviral vectors, or particles, nanoparticles or liposomes, which contain the polynucleotide. The polynucleotide may also be incorporated into the genome of the cell. The sequence of the polynucleotide may be optimized according to the most favorable codon usage of the cell used. The sequence may also include additional regulatory elements which increase or reduce the stability of the polynucleotide in the cell.

In order to generate a relatively large amount and/or, as described above, a particular hierarchic structure of the organic-inorganic composite material, the host cell is conveniently cultured, i.e. propagation of the cell is stimulated, where appropriate prior to expression, and this may also result in differentiation of the host cell or of some of the propagated host cells to a particular tissue or a cell type.

In an advantageous embodiment of the invention, the expressed protein is incorporated into the extracellular matrix of the host cell.

In another advantageous embodiment, the inorganic particles are formed only in the extracellular matrix, in particular due to incorporation of the expressed protein. This may optionally also be supported or promoted by adding precursors of said inorganic particles to the host cell and/or the extracellular matrix. Said precursors may be added before, during and/or after expression. Preference is given to treating the extracellular matrix with the precursors. Depending on the expressed protein and/or polypeptide, the precursors may be solutions of salts such as calcium chloride, for example, or chemical precursors such as alkoxysilanes, for example. It is also possible to add only some of the substances required for generating the inorganic particles, for example particular cations or anions. A combination of precursors may also be employed. The solutions may also contain complexed cations or anions. Preference is given here to halides (e.g. fluorides, chlorides, bromides, iodides), sulfates, phosphates, hydroxides, sulfides, carbonates, hydrogencarbonates, salts of carboxylic acids (e.g. citrates, oxalates, tartrates or malates) with metals or metalloids of groups 1 to 16 of the Periodic Table, particularly preferably Li, Ca, Mn, Fe, Zr, Ti, Ba, Si, Al, Zn, Sr, Mg, Mo, Co, Ni, Ag, Au, Ga, Se or Cu. Organic cations such as ammonium ions may also be included. Mixtures of precursors may also be employed.

In another embodiment of the method, the inorganic particles are formed in the host cell and then incorporated into the extracellular matrix of the host cell. This may also require the addition of precursors according to the information above.

The expressed protein and/or polypeptide encoded by the recombinant polynucleotide may be a protein which is xenogenous (heterologous) or homologous to the host cell, preferably a protein xenogenous to the host cell. It may also be only part of a protein.

The protein and/or polypeptide encoded by the polynucleotide may be any proteins and/or polypeptides. Preference is given to sequences which contain at least one of the structures listed in table 1 (the + signs in table 1 indicate the strength of the interaction of the structures).

The protein and/or polypeptide may also be one that has at least 80%, 90%, preferably 95%, 99% or 100% sequence homology with a protein and/or polypeptide with biomineralizing action from an animal, a plant, a fungus or a bacterium. Such proteins preferably belong to the class of hydrolases (EC Class 3), transferases (EC Class 2) or oxidoreductases (EC Class 1) or lectins. They may, however, also have particular sequences that can bind ions or can form polar bridges, for example the sequences from table 1.

In an advantageous embodiment of the invention, the protein and/or polypeptide is selected from table 2. Preference is given to proteins and/or polypeptides with at least 80%, 90%, preferably 95%, 99% or 100% sequence homology with a protein, as listed in table 2. Said sequence homology also refers to the DNA sequences indicated in the table with regard to the amino acids encoded by them. They may also be only part of the proteins mentioned above.

In one embodiment of the invention, the encoded protein is selected from the group comprising Ansocalcin, ovocleidin-17, perlucin, SM32, N16.1, silicatein a, silicatein b, nacrein, Lustrin A, amelogenin and enamelin. Examples of these enzymes or partial sequences thereof are listed in table 3. Particular preference is given to Ovocleidin-17, N16.1 and Perlucin or partial sequences thereof. Preferred sequences are indicated in table 3, in particular the protein sequences indicated there.

In an advantageous embodiment, the introduced polynucleotide includes at least one noncoding regulatory section called promoter for controlling expression of the coding section for the protein and/or polypeptide. In particular, the promoter enables expression of the encoded protein and/or polypeptide to be restricted to particular cells or to be coupled to a particular differentiation of the cell. By specifically selecting or changing the promoter sequence, expression of the protein can be coupled to environmental parameters such as ionic strength or temperature, and chemically inhibited or induced by the absence or addition of special substances. This controlling option is essential, since the encoded protein may under certain conditions be damaging to the cell and/or a biomineralizing action may likewise be dependent from environmental parameters such as ionic strength or temperature and may be dependent due to the absence or addition of special substances.

In an advantageous embodiment, the recombinant polynucleotide additionally contains at least one nucleotide sequence section (referred to as coding for the signal peptide hereinbelow) which encodes an amino acid sequence for controlling localization and/or time of expression of the protein and/or polypeptide. Said nucleotide sequence may encode a polypeptide which is completely or at least 80%, 90%, preferably 95% or 99%, homologous to an amino acid sequence of the host cell. It may however also be a xenogenous or synthetic amino acid sequence. It may also be only part of such a polypeptide. Particular preference is given to an amino acid sequence that is completely homologous to a polypeptide from the host cell. The amino acid sequence is preferably a signal sequence. Such sequences are known to the skilled worker from a multiplicity of organisms and allow, for example in the case of plants, expression and/or localization in particular plant parts. Particularly important here is the specific distinction between the noncoding promoter polynucleotide sequence and the polynucleotide sequence coding for the signal peptide, i.e. the polynucleotide sequence intended for translation. Both sequences are selected and adapted to the desired subsequent biomineralizing action of the protein according to in each case different criteria, with the different developmental-biological and cell-physiological conditions in the corresponding host organisms being taken into account.

Signal sequences or signal proteins are usually peptides or amino acid sequences which determine the destination or site of expression of a protein inside a cell. Proteins whose destination is outside the cell, in biomembranes or in compartments, typically possess signal sequences. Thus, for example, transport into the extracellular space or the cell membrane can be induced as a function of the signal sequence. After transport or when passing through a membrane or being incorporated into a membrane, the signal sequence may be processed, typically cut off, for example by a signal peptidase. In the secreted state, the protein may be either with or without signal sequence. The presence of the signal sequence need not necessarily influence the functionality of the protein with biomineralizing action. It may be a signal sequence for secreting the expressed protein into the extracellular matrix of the host cell. In the case of higher organisms, it may also be a signal sequence for expressing and/or incorporating the expressed protein into the cell wall of the host cell. This is preferred in the case of fungal and plant cells.

Secretion for the purposes of the application means the transport or addition of the expressed protein and/or polypeptide into a compartment outside the cell membrane delimiting the cytoplasma or into the extracellular matrix.

Advantageously, the recombinant polynucleotide encodes the at least one protein and/or polypeptide and the amino acid sequence for controlling localization and/or time of expression of said protein and/or polypeptide in such a way that both are fused to one another in the translation product. It is also possible here for up to 30 nucleotides to be inserted between the two sequences on the polynucleotide. Preference is given to the nucleotide sequence of the signal sequence being inserted upstream of the nucleotide sequence of the protein and/or polypeptide in the direction of transcription. It is also possible for a functional protein domain belonging to the protein (e.g. the normal case for transmembrane proteins) to control localization. As a result, the amino acid sequence for influencing secretion is located at the N-terminal end of the fusion protein. It could be advantageous to provide the protein as transmembrane protein preferably with a correctly oriented membrane insertion domain rather than its own signal sequence, in particular if an interaction with intracellular signal pathways is desired. In this case the intracellular control domain would be located on the N terminus.

Suitable signal sequences and transmembrane sequences are known to the skilled worker, depending on the host cell used.

Examples of signal sequences or proteins containing such sequences can be found in table 4, with preference being given to Ecmb or the signal peptide therefrom.

Preferred fusion proteins contain sequences having at least 80%, 90%, preferably 95%, 99% or 100%, sequence homology to a fusion protein of a protein from table 3 with signal peptides from table 4, with preference being given to Ecmb or the signal peptide therefrom.

The host cell is a cell from the class of slime molds (Eumycetozoa), preferably of the genus Dictyostelium, more particularly of the species Dictyostelium discoideum.

These are host cells which can be induced to assemble to higher order multicellular units. This is the case for Dictyostelium discoideum, for example. Depending on the developmental state of the slime mold, it is possible to simulate in a simple manner different conditions for the action of the protein. A suitable choice of promoter enables expression to be targeted to a particular developmental state.

In the case of Dictyostelium, an outer sheath of cellulose and glycoproteins forms during development of the mold stalk. Said sheath shows similarities to the membranes of plant cells. Efficacy may also be investigated in a simple manner in very different cellular stages (living cells/cell aggregates/resting cells/moving cells/dying cells/dead cells). Therefore it is possible to test at the same time various biomineralizing actions of one and the same protein of unknown action as a function of the limiting conditions.

Expression of the protein and/or polypeptide encoded on the recombinant polynucleotide may be coupled to a change in culturing conditions, the addition of a substance and/or to a particular state of the host cell. A state here may be a particular phase of cell development and/or a response to an external or internal stimulus such as, for example, a particular differentiation of the host cells which results in synthesis of the extracellular matrix that forms the organic component of the organic-inorganic composite material. Expression may therefore be limited to particular host cells, preferably to the host cells producing said extracellular matrix. Preference is given to expression being controlled according to the conditions indicated above with the aid of a promoter suitable for the host cell. Promoters of this kind are known to the skilled worker from the prior art.

Moreover, the expressed protein and/or polypeptide may be modified post translation, for example by glycosidation, phosphorylation, acetylation, alkylation, hydroxylation or the like. These modifications may alter and/or (even) cause the biomineralizing property of the protein and/or polypeptide.

Like expression, secretion of the expressed protein and/or polypeptide may be coupled to a change in culturing conditions, the addition of a substance and/or to a particular state of the cell, for example when the cell attains a particular differentiation which induces synthesis of the extracellular matrix. Secretion may therefore be limited to particular cells. Preferably to the cells which form the extracellular matrix. This is enabled, for example, through the choice of signal peptide and/or the corresponding promoter. Advantageously, expression and secretion are triggered by two different signals.

The biomineralizing action of the protein and/or polypeptide may be investigated in various ways. For example, the addition of precursors for inorganic solids may be required during culturing. It may also be necessary to reduce or to enhance particular other metabolic processes in the host cell, for example in order to provide a higher concentration of precursors. The host cell may also contain polynucleotides encoding further recombinant proteins, or further xenogenous, recombinant proteins and/or polypeptides, in particular proteins and/or polypeptides with biomineralizing action. For example, these proteins may also modify the extracellular matrix and/or modify the interaction of the protein and/or polypeptide with biomineralizing action.

Moreover, it may also be necessary for the organic-inorganic composite material to be treated at least once with at least one precursor for the formation of an inorganic material, for example in order to generate particular morphologies or structures. This may be utilized, for example, for altering the size of the particles formed or to apply a further layer of a different inorganic solid, which may also be a different morphology. Said treatment may be carried out using living cells. It is also possible, however, for the host cells to be dried or lyophilized, for example, and then for a test, optionally further tests, of the biomineralizing action to be carried out.

In an advantageous embodiment of the invention, investigating the biomineralizing action comprises contacting the host cell with a solution of at least one salt. Preference is given here to solutions of salts such as, for example, calcium chloride, or to chemical precursors such as alkoxysilanes, for example. The solutions may also contain complexed cations or anions. Preference is given here to halides (e.g. fluorides, chlorides, bromides, iodides), sulfates, phosphates, hydroxides, sulfides, carbonates, hydrogencarbonates, salts of carboxylic acids (e.g. citrates, oxalates, tartrates or malates) with metals or metalloids of groups 1 to 16 of the Periodic Table, with particular preference being given to Li, Ca, Mn, Fe, Zr, Ti, Ba, Si, Al, Zn, Sr, Mg, Mo, Co, Ni, Ag, Au, Ga, Se or Cu. Organic cations such as ammonium ions may also be included. Mixtures of precursors may also be employed. Preferred compounds are chlorides, carbonates or sulfates, preferably of Li, Ca, Fe, Ba, Zr or Ti.

After optional treatment of the host cell (drying, freeze drying), the biomineralizing action of the expressed protein may be investigated in various ways. Thus it is possible, for example, to test optical properties such as refractive index or specific rotation, or else the content of inorganic substances. The samples may also be tested for particular crystalline structures by light microscopy examinations, for example using polarized light. Similarly, other methods such as Raman spectroscopy may also be used. Optical methods in particular are especially suitable for screening large libraries.

In one embodiment of the invention, the method is carried out using a library of recombinant polynucleotides. Such a library may, for example, be a library of a protein with biomineralizing action that has been prepared by error-prone PCR. Because the method is easy to carry out, in particular when using mold cells, particularly preferably of Dictyostelium, it is possible in this way to investigate a multiplicity of proteins with a potentially biomineralizing action and to test them for their action in a multicellular organism. The results obtained by this method regarding advantageous sequences may then be transferred to higher organisms, the culturing of which is frequently significantly more complex. At the same time it is also easier to test biomineralizing conditions. The starting proteins used are preferably proteins of table 2, particularly preferably of table 3.

Proceeding from the properties found, a decision is made as to whether a further optional stage of the method is carried out using this protein.

In a further advantageous embodiment of the invention, the method, after it has been carried out, preferably after identification of biomineralizing sequences with the aid of said method, is carried out again in a second host cell.

For this, a second recombinant polynucleotide is introduced into a second host cell, said polynucleotide encoding the protein and/or polypeptide from the first stage of the method, and said polynucleotide being suitable for expression of the encoded protein and/or polypeptide in the second host cell. Said second host cell is then cultured and the biomineralizing action of the expressed protein is triggered. These steps of the method are also referred to as second stage (stage II) hereinbelow. The previous identification of the protein is referred to as first stage (stage I).

The second recombinant polynucleotide encodes the protein used when the method was carried out for the first time. It may be necessary here to adapt the sequence of the polynucleotide to the codon usage of the second host cell.

If a preselection of sequences for proteins with potentially biomineralizing action, which has been obtained by the method of the invention, is already available for this stage II of the method (e.g. table 2), the host cells can rapidly be alternated by appropriately choosing the cloning system (prior art: e.g. Gateway cloning and by means of PCR). This rapid alternation is enabled by the availability of a universally usable system for investigating the biomineralizing action, which system consists of the first host organism with a defined structure of the extracellular matrix as the site of action for the protein/polypeptide/matrix combinations to be tested, and a corresponding suitable promoter-signal peptide-Gateway cassette system. It is also important for the functionality of the system that the host organism can be induced to assemble to higher order multicellular units.

Advantageously, the second recombinant polynucleotide likewise comprises a signal sequence for influencing localization and/or controlling expression in the second host cell. The second polynucleotide preferably encodes a fusion protein of the protein with biomineralizing action and a signal sequence suited to the second host cell, optionally with a short linker of from 3 to 18 nucleotides being located between the two sequences. If a fusion protein was used when the method was carried out for the first time, then it is possible for the second recombinant polynucleotide used in the second procedure to have a different signal sequence. This may be necessary in order to influence localization and/or expression of the encoded fusion protein in the second host cell.

All fusion proteins in the method may of course still have further sequences with particular properties. These may be, for example, tags encodable by amino acids or labels. The latter may be fluorescent labels, for example GFP (green fluorescent protein). They may also be affinity labels such as His tag, HA tag, streptavidin or similar tags. Combinations of tags may also be employed. Such labels may be utilized for analyzing localization and expression of the proteins.

In one embodiment of the invention, the host cell used in the second stage of the method is a plant cell, for example selected from the group comprising: Arabidopsis (Arabidopsis thaliana, Thellungiella Halophila), tobacco, fiber-producing plants (bamboo, flax (Linum), hemp, cotton, jute (Corchorus), sisal agave (Agave sisalana), coconut palms), grasses (rice, corn, barley, wheat, millet, miscanthus), woody plants (opus, eucalyptus, pines, Pinaceae), preferably from the group comprising Arabidopsis (Arabidopsis thaliana, Thellungiella halophila), tobacco, fiber-producing plants (bamboo, flax (Linum), hemp, cotton, jute (Corchorus), sisal agave (Agave sisalana), coconut palms).

The second polynucleotide may be introduced using any techniques known to the skilled worker, depending on the second host cell. For example, the second polynucleotide may have sequences which allow integration into the genome of the second host cell, for example via T-DNA insertion or homologous recombination. However, transfection methods such as PEG-mediated transfection or bombardment methods which are used for grasses (Poaceae, monocots), for example may also be utilized.

If the second host cell is a plant cell, it is also possible to select only some of the cells of the chosen plant as host cells, for example the leaves or the roots.

The invention furthermore relates to an organic-inorganic composite material which contains an extracellular matrix of at least one host cell as the organic component, with inorganic particles having been incorporated into the extracellular matrix of said host cell by expression of at least one recombinant protein and/or polypeptide with biomineralizing action. Said protein and/or polypeptide may also be part of the organic component of the composite material.

In an advantageous embodiment, the at least one expressed protein and/or polypeptide is xenogenous to the host cell.

The expressed protein and/or polypeptide may be a protein and/or polypeptide which has at least 80%, 90%, preferably 95%, 99% or 100%, sequence homology to a protein and/or polypeptide with biomineralizing action from an animal, a plant, a fungus or a bacterium. Preference is given to proteins and/or polypeptides having at least 80%, 90%, preferably 95%, 99% or 100%, sequence homology with a protein as listed in table 2. Particular preference is given to proteins having at least 80%, 90%, preferably 95%, 99% or 100%, sequence homology with the proteins from table 2, preferably from table 3.

In one embodiment of the invention, the expressed protein and/or polypeptide is a fusion protein of at least one protein and/or polypeptide with biomineralizing action and an amino acid sequence for influencing secretion of the fusion protein into the extracellular matrix. The amino acid sequence may be a polypeptide which is completely or at least 80%, 90%, preferably 95%, 98%, 99%, or completely homologous to a polypeptide of the host cell. It may also be a xenogenous polypeptide, however. Preference is given to a polypeptide which is at least 80%, 90%, preferably 98%, 99%, or completely homologous to a polypeptide of the host cell. It may also be only part of such a polypeptide. Particular preference is given to a polypeptide which is completely homologous to a polypeptide from the host cell. The peptide is preferably a signal peptide for influencing localization and/or expression. The signal peptide is preferably located at the N-terminal end of the fusion protein. It is also possible for further amino acids to be inserted between the signal peptide and the biomineralizing protein and/or polypeptide.

In one embodiment of the invention, the organic-inorganic composite material is obtainable by the method described above. The composite material can be obtained after stage I or stage II.

In a preferred embodiment, the organic-inorganic composite material possesses a hierarchic structure, preferably an anisotropic hierarchic structure.

Advantageously, the organic component of the organic-inorganic composite material, depending on the host cell, essentially consists of a biopolymer, preferably selected from the group comprising polysaccharides or filamentous proteins such as, for example, cellulose or starch, lignin, collagen, lipids, polyglucosamines such as chitin or chitosan, pectins or their derivatives, particularly preferably polysaccharides such as cellulose and their derivatives. The organic component may also contain combinations of a plurality of different biopolymers.

The proportion of the inorganic component may be between 1 and 98% by weight of dry matter of the organic-inorganic composite material, preferably between 2% by weight and 50% by weight.

The inorganic particles may have a maximum diameter of between 3 and 10 000 nm, preferably between 20 and 1000 nm, particularly preferably between 200 and 500 nm. They may have an amorphous, semi-crystalline or crystalline morphology. Preference is given to semi-crystalline or crystalline particles, particularly preferably crystalline particles. The particles may have any shape, for example platelet-like shapes are also possible. Advantageously, the particles have a spherical or angular shape.

In one advantageous embodiment of the invention, the particles include oxides, hydroxides, carbonates, phosphates, fluorides, sulfides, sulfates and/or salts of carboxylic acids such as citrates, oxalates, tartrates or malates, more particularly those compounds with metals or metalloids of groups 1 to 16 of the Periodic Table, preferably Li, Ca, Mn, Fe, Zr, Ti, Ba, Si, Al, Zn, Sr, Mg, Ba, Mo, Co, Ni, Ag, Au, Ga, Se or Cu. This also includes compounds with oxide anions such as titanates or tungstenates. Preference is given here to particles of iron oxide (FeₓO_{y}), manganese oxide (MnₓO_{y}), silicon dioxide, silicates, copper oxide (Cu₂O), iron sulfide (FeₓS_{y}), calcium carbonate and/or calcium phosphate, with all compounds optionally both being hydrated and containing proportions of further cations such as, for example, alkali metal or alkaline earth metal ions, or other anions such as, for example, halide ions. Examples of formed minerals are calcite, vaterite, aragonite, hydroxylapatite, fluorapatite or magnetite.

Another aspect of the invention relates to a recombinant nucleic acid which encodes a fusion protein of at least one protein and/or polypeptide with biomineralizing action or parts thereof and at least one amino acid sequence, preferably a signal peptide, for influencing secretion of said fusion protein, and includes a corresponding promoter. The nucleic acid may be in the form of a DNA, cDNA, RNA or mixtures thereof. The nucleic acid may also include one or more introns and/or may be part of a vector. Advantageously, the nucleic acid also includes a promoter for the encoded fusion protein. The nucleic acid may also encode only parts of said proteins, polypeptides and/or amino acid sequences.

Advantageously, the encoded protein and/or polypeptide has at least 80%, 90%, preferably 95%, 99% or 100%, sequence homology to a protein and/or polypeptide as depicted in table 2 or preferably in table 3. The method described comprises identifying in stage I out of a pool of very different proteins and/or polypeptides with potentially biomineralizing action or of the corresponding polynucleotide sequences specifically those which have biomineralizing action.

Another aspect of the invention relates to a recombinant protein and/or polypeptide comprising a fusion protein of at least one protein and/or polypeptide with biomineralizing action and at least one amino acid sequence, preferably a signal peptide, for influencing secretion of said fusion protein. Preference is given to a protein and/or polypeptide having at least 80%, 90%, preferably 95%, 99% or 100%, sequence homology to a protein and/or polypeptide from table 2, preferably from table 3, and including an amino acid sequence for controlling secretion of said protein and/or polypeptide. It may also contain only parts of the proteins and/or polypeptides from table 2, preferably from table 3. The amino acid sequence may be at least 80%, 90%, preferably 95%, 99% or 100%, homologous to the sequence of a protein and/or polypeptide from the host cell, but it may also be a synthetic peptide, with preference being given to a completely homologous amino acid sequence in respect of the biomineralizing donor organism or the host cell (acceptor organism, genetically modified organism, etc.), but for the signal sequence in respect of the host cell. The signal sequence is not taken into account for calculating homology.

In one embodiment of the invention the signal sequence has at least 80%, 90%, preferably 95%, 99% or 100%, sequence homology to a signal sequence from table 4.

Advantageously, the amino acid sequence resulting in the specific location in the tissue of the target organism is located on the N terminus of the fusion protein. It is possible for also further, up to 10, amino acids to be inserted between the two sequences.

Another aspect of the invention relates to a host cell, preferably from the class of slime molds (Eumycetozoa), which contains a nucleic acid according to the invention. The host cell may be obtained, for example, by being transfected, infected or transduced, for example by treatment with plasmids, phagemids, cosmids, retroviral or adenoviral vectors, or particles, nanoparticles or liposomes, which contain the nucleic acid according to the invention. Methods of this kind are known to the skilled worker. The host cell may also belong to any of the organisms mentioned for stage II.

Another aspect of the invention relates to the use of the recombinant nucleic acid of the invention for producing an organic-inorganic composite material of the invention.

The organic-inorganic composite materials of the invention can be used in various ways, depending on their properties. Since they are based on natural processes, they can be produced with low energy consumption, in particular in the presence of an appropriate host cell capable of independent reproduction (e.g. for plants in the form of seeds).

An important property is the increased strength and hardness as a result of said incorporation. This involves modified woods, for example, to be used as building material. Fibers produced from such a composite material may also be used, for example, for ropes, textiles and the like. Crushed organic-inorganic composite materials may also serve as additives for coatings. It is also possible for the optical properties of small particles to be utilized in order to confer novel optical properties on these materials. This may also result in an advantage in that the inorganic particles can be removed by treatment with aqueous solutions, with novel properties being produced due to the resulting cavities (e.g. lightweight material).

The organic matrix may also be removed by thermal treatment, for example, thus yielding an inorganic material having a particular morphology and/or structure.

Further details and features arise from the description below of preferred exemplary embodiments in conjunction with the dependent claims. The particular features may be implemented here on their own or as a plurality thereof in combination with one another. The possible solutions to the object are not limited to said exemplary embodiments. Thus, for example, range information always comprises all intermediate values (not mentioned) and all possible subintervals.

Table 3 lists sequences for various proteins. N16.1 or N16N has the native protein sequence Seq. ID No. 5. The DNA sequence Seq. ID 19 encodes a partial sequence of the protein containing a tag. The protein has been slightly modified in this case. Seq. ID 78 encodes this partial sequence of the protein already optimized for the expression system used. For OC-17, Seq. ID No. 2 depicts the protein sequence of the protein. Seq. ID No. 79 is the DNA sequence protein already optimized for the expression system used. Seq. ID No. 20 depicts the DNA sequence of a slightly modified OC-17 protein. Seq. ID No. 80 has also been optimized for the expression system used.

### Methods

### Recombinant DNA Technology

To manipulate DNA, use was made of standard methods as described in Sambrook, J. et al. (1989) In. Molecular cloning: A Laboratory Manual.

The enzymes were obtained from commercial sources and employed according to the manufacturers' instructions.

### Cloning System

The vectors were designed on the basis of the Gateway cloning system. The target sequence is introduced from an entry vector into the destination vector using the LR Clonase reaction. This involves recombination between entry vector with attL1 and attL2 sites and the destination vector with attR1 and attR2 recombination sites.

### BP/LR Reaction Procedure:

First, 7 μl of PCR product or entry vector with 1 μl of destination vector were introduced into an Eppendorf vessel. The Clonase was briefly agitated, and 2 μl were added to each reaction mix. Recombination was carried out at 25° C. overnight. The reaction was stopped by adding 1 μl of proteinase K to the reaction mix and incubating at 37° C. for 10 minutes.

### Expression in Dictyostelium

### Construction of the Vector

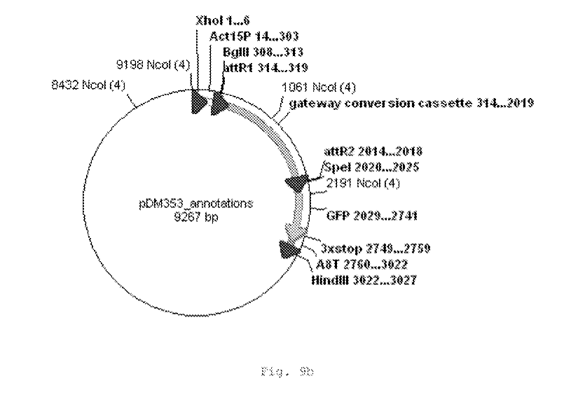

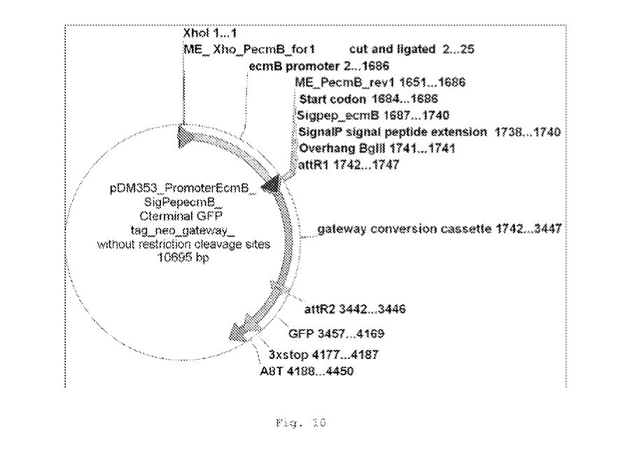

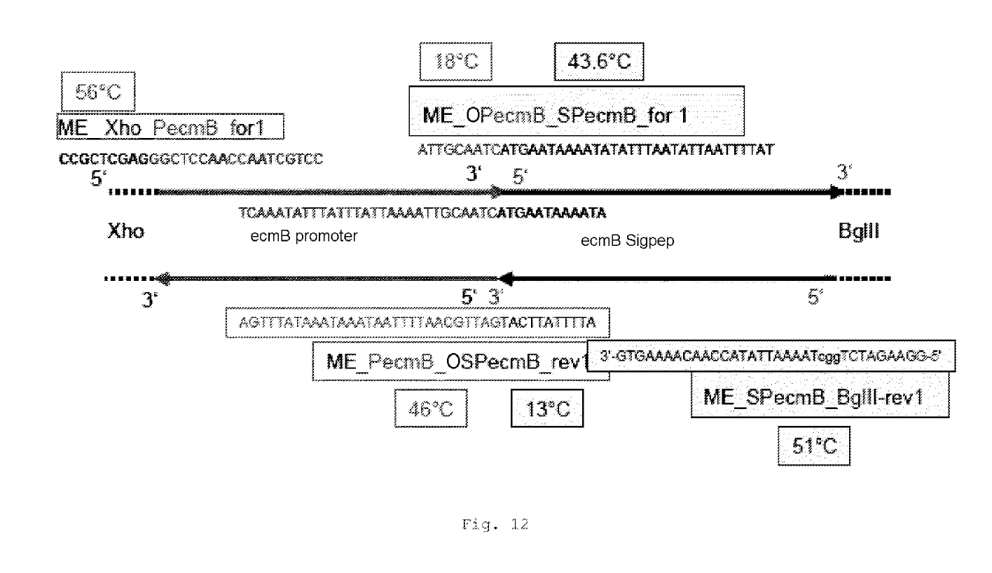

To introduce the proteins into the cell, the vectors pDM353 (Seq. ID No. 33; Veltman, D. M., Akar, G., Bosgraaf, L.& Van Haastert, P. J. M. A new set of small, extrachromosomal expression vectors for Dictyostelium discoideum. Plasmid 61, 110-118 (2009); Veltman, D. M. Extrachromosomal expression vector. Gateway. G418 resistance. C terminal GFP tag. http://dictybaseorg/db/cgi-bin/dictyBase/SC/plasmid detailspl?id=546 (2009). FIG. 9a, FIG. 9b) and EcmB-Gal (Jermyn, K. A.& Williams, J. G. An analysis of culmination in Dictyostelium using prestalk and stalk-specific cell autonomous markers. Development 111, 779-87 (1991); Williams, J. G. Galactosidase fusion expression vector (prestalk marker). http://dictybase.org/db/cgi-bin/dictyBase/SC/plasmid_details.pl?id=51 (1991). FIG. 11; Seq. ID 83) from DictyStockCenter (Fey, P. et al. dictyBase—a Dictyostelium bioinformatics resource update. Nucleic Acids Research 37, D515-519 (2009); www.dictybase.org. About Dicty Stock Center (DSC)) were obtained and used as templates for the subsequent steps. Furthermore, the EcmB gene may also be derived from the pDd56 vector.

EcmB was also derived from the EcmB-Gal vector by PCR cloning (K. A. Jermyn, J. G. Williams, Development 1991, 111, 779). The primers “ME-XhoI_PecmB_for2” (Seq. ID No. 69) and “ME_PecmB_Nco_rev” (Seq. ID No. 70) were used for this.

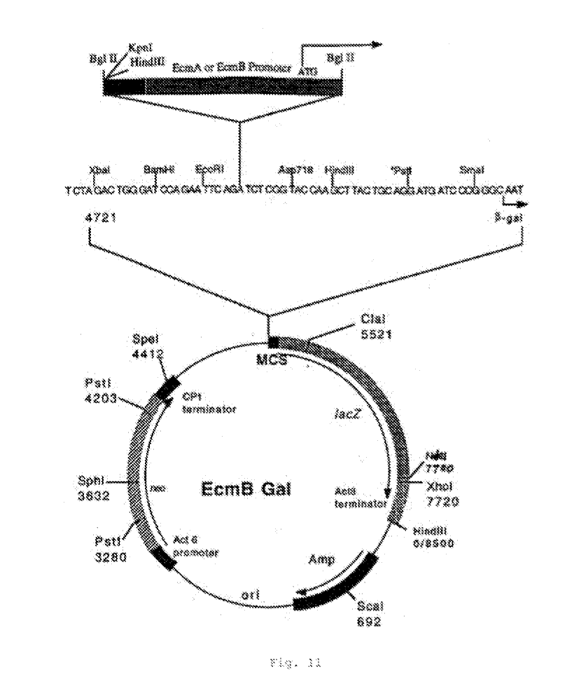

PCR cloning was used for introducing XhoI and BglII restriction sequences which delimit the actin15 promoter of pdM353 into a DNA fragment (“ME_ecmB_SigP_for” Seq. ID No. 71) which contains the EcmB promoter, an NcoI restriction sequence, the Kozak sequence, the ATG start codon and part of the signal peptide of the ecmb gene product (FIG. 10). The ecmB promoter and the DNA fragment were ligated by means of PCR (Primer: “ME_Xho_PecmB_for2” (Seq. ID No. 69) and “ME_ecmSP_Bgl_rev” (Seq. ID No. 72). The purified DNA (with Xho and BGlII cleavage sites) was phosphorylated with T4 polynucleotide kinase and, at a ratio of 4:1, cloned into a SmaI-cut, dephosphorylated pBluescript SK-vector (Strategene) and sequenced. This produced a stable vector. Positive clones were detected by colony PCR (Primers: ME_Xho_PecmB_for2″ and “ME_ecmSp_Bgl_rev”).

The Gateway destination vector was obtained by conventionally cloning the actin15 promoter-containing pDM353 and the pBluescript SK-containing the <EcmB promoter . . . signal peptide> subregion prepared above. The promoter of the vector was replaced using the XhoI and BglII restriction sites. The modified vector was transformed into E. coli. Positive clones were detected by colony PCr (“ME_Xho_PecmB_for2” and “Me_ecmSP_Bgl_rev”).

The method presented and the availability of the pDM vectors (Veltman, D. M., Akar, G., Bosgraaf, L.& Van Haastert, P. J. M. A new set of small, extrachromosomal expression vectors for Dictyostelium discoideum. Plasmid 61, 110-118 (2009)) allow fusion proteins with labels such as GFP or immunoaffinity labels to be prepared in a simple manner.

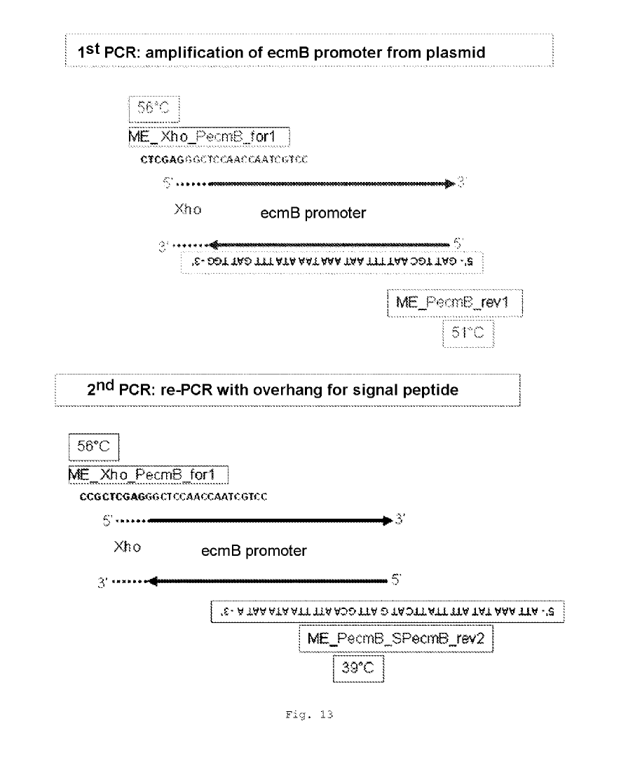

The vector construct is depicted diagrammatically in FIG. 10. The cloning strategy of individual components based on the ecmb signal sequence is depicted in FIGS. 12 (Seq. ID No. 39, 40), 13, 14, 15 (Seq. ID No. 25, 26, 27, 28, 29, 30), 19 (Seq. ID No. 73, 74). The primers are listed again in table 5.

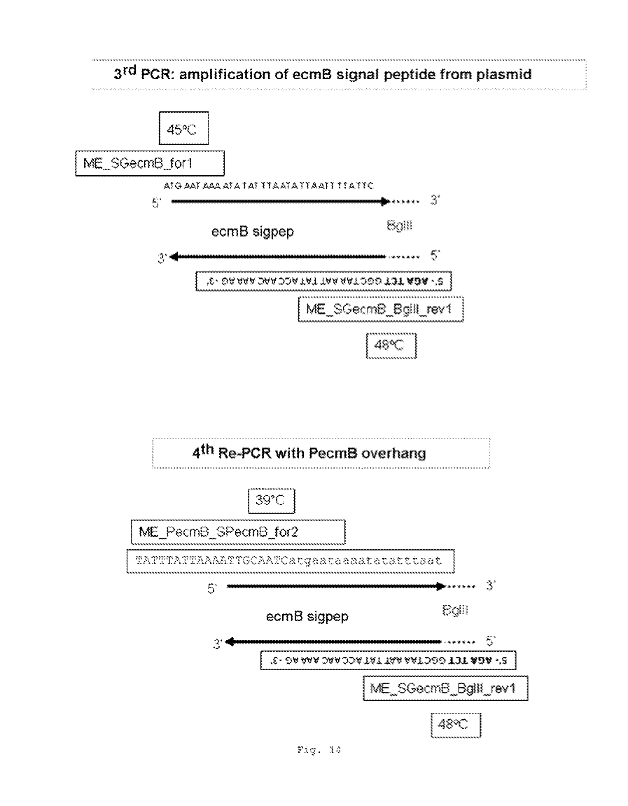

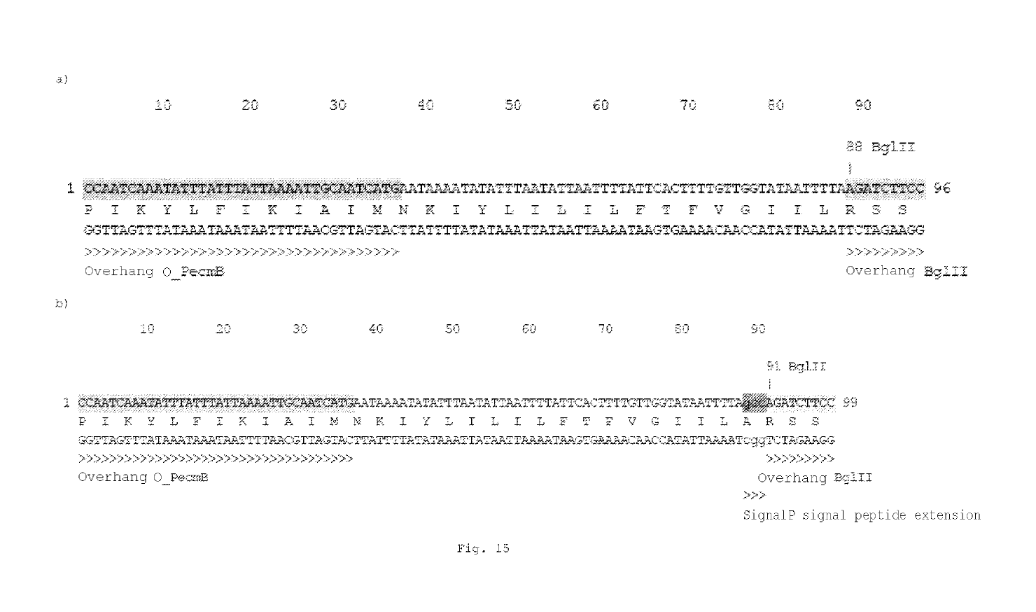

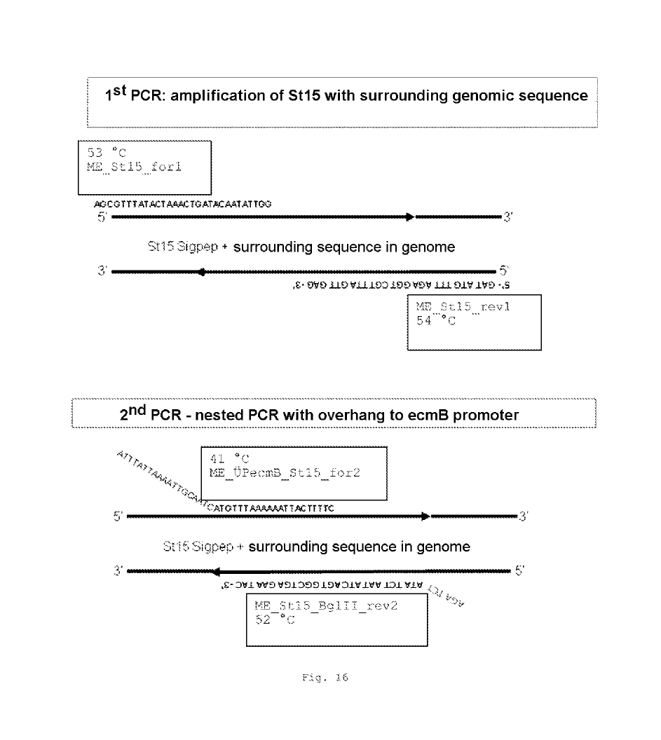

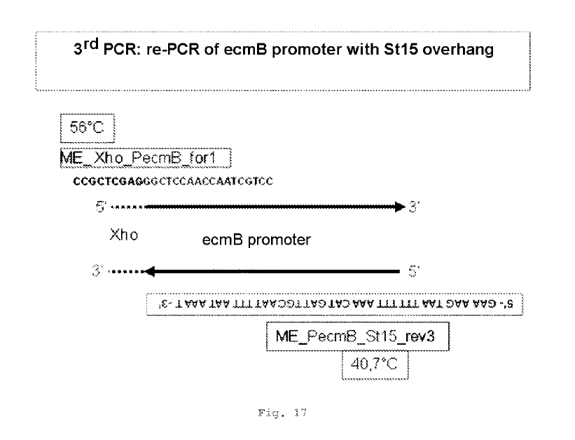

Additionally, the vector may also contain a cellulose-binding domain (St15). Further information on this can be found in FIGS. 16, 17, 18 and 20. This domain also contains a signal peptide which may likewise be used. Further primers are listed in table 5 and can readily be assigned on the basis of their name.

The work was carried out using standard protocols. Further primers can be found in table 5. The signal sequences were extended by different cleavage sites and in some cases also by an amino acid (signal peptide extension) and then introduced into the vector.

Production of the proteins with biomineralizing action The following protocol may be used generally for different proteins for Dictyostelium:

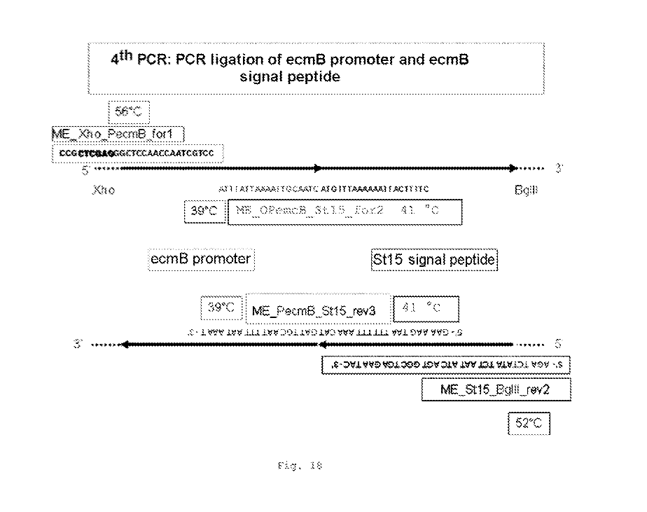

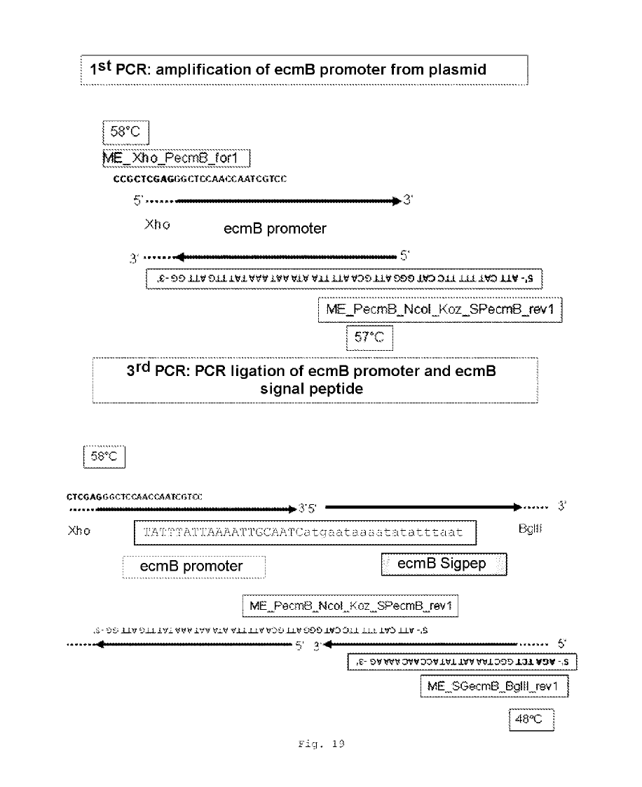

The protein sequences were “back-translated” bioinformatically into DNA sequences, taking into account codon usage parameters, with the aid of Leto software (Entelechon, Regensburg, Germany). The corresponding synthetic genes were purchased from Entelechon and used as starting point for PCR cloning. This is depicted by way of example in FIG. 21 (Seq. ID 75, 76, 77) for perlucin. Further information can be found also in the codon usage table in Nucleic Acids Research 2000, vol. 28, no. 10, Vervoot et al., “Optimizing heterologous expression in dictyostelium: importance of 5′ codon adaptation” to which reference is made hereby.

A synthetically produced gene (pENTR/D-TOPO_SP-perlucin_opt, Entelechon, Regensburg, Germany), encoding the nacre-specific C-type lectin biomineralization protein perlucin (Swiss-Prot: P82596.3) was amplified using the following primers: (“DreamTag” DNA polymerase Fermentas PCR-extension at 68° C.)

Primer 1, forward ME_CACC_Per_for_1a
(Seq. ID No. 31):
CACCGGATGTCCTTTGGGTTTTCACC
Primer 2, reverse EW_Per_rev_1
(Seq. ID No. 32):
TCTTTGTTGCAGATTGGCGTGAAGC

Template: pENTR/D-TOPO_SP_perlucin_opt (Entelechon)

The PCR product was cloned into a pENTR/D-TOPO vector (FIG. 9) with the aid of the Stratagene Gateway cloning kit (Invitrogen. pENTR™ Directional TOPO® Cloning Kits—Five-minute, directional TOPO® Cloning of blunt-end PCR products into an entry vector for the Gateway® System. User Manual Version G, 25-0434 (2006)). One of the following cell lines was used:
One Shot® E. coli cells (TOP10: F-mcrA Δ (mrr-hsdRMS-mcrBC) Φ801acZΔM15 ΔlacX74 recA1 araD139 Δ(ara-leu) 7697 galU galK rpsL (Str^{R}) endA1 nupG; or
Mach1™-T1^{R}: F-Φ801acZΔM15 ΔlacX74 hsdR (rₖ⁻, mₖ⁺) ΔrecA1398 endA1 tonA (confers resistance to phase T1)

The cells were transformed, selected and analyzed. The cells containing the pENTR/D-TOPO vector were used in the LR reaction with the Gateway destination vector prepared above with the aid of the Gateway® LR Clonase™ II enzyme mix (Stratagene).

### Other Sequences:

In addition, the genes for n16N (GeneBank No. AB023251.1), OC-17 (Swiss-Prot: Q9PRS8.2) and perlucin (GeneBank No. FN67445.1) were used.

These genes were extended by the sequences required for cloning via PCR using the primers ME_CACC_Per_forla (Seq. ID No. 31), EW_Per_rev_{—}1 (Seq. ID No. 32), ME_N16N_for1 (Seq. ID No. 65), Ext3-N16N_Rev (Seq. ID No. 66), ME_OC17_for1 (Seq. ID No. 67), Ext3_OC17_Rev (Seq. ID No. 68). The sequences were introduced into pENTR/D-TOPO vectors using the methods described previously. Incorporation was checked by PCR using the appropriate primers.

The genes were introduced via the LR reaction into the pDM353 ecmB_SigP_att_GFP vector obtained previously and cloned into E. coli. The clones were selected with ampicilin and the presence of the gene was checked by Colony PCR. All of the E. coli selected contained a pDM353_ecmB_SigP_X_GFP Dictyostelium expression vector (X is n16N, OC17 or perlucin).

The vectors obtained were transformed into Dictyostelium discoideum cells AX3-ORF⁺ (Manstein, D. J., Schuster, H.-P., Morandini, P.& Hunt, D. M. Cloning vectors for the production of proteins in Dictyostelium discoideum. Gene 162, 129-134 (1995)) with the aid of methods known in the literature (Nellen, W., Silan, C. & Firtel, R. A. DNA-mediated transformation in Dictyostelium discoideum: regulated expression of an actin gene fusion. Mol Cell Biol 4, 2890-8 (1984); Pang, K. M., Lynes, M. A. & Knecht, D. A. Variables Controlling the Expression Level of Exogenous Genes in Dictyostelium. Plasmid 41, 187-197 (1999); Gaudet, P., Pilcher, K. E., Fey, P. & Chisholm, R. L. Transformation of Dictyostelium discoideum with plasmid DNA. Nat. Protocols 2, 1317-1324 (2007); www.dictybase.org. Transformation of Dictyostelium by calcium phosphate precipitation. http://dictybase.org/techniques/transformation/calcium phosphate.html (2010); www.dictybase.org. Transformation of Dicty by electroporation. http://dictybase.org/techniques/transformation/knecht electroporation protocol.html (2010)). Examples of suitable methods are electroporation or chemical methods. Individual clones were obtained by several selection steps in the presence of G418 (10-100 μg/ml) and cultured to a cell density of 1×10⁹ ml⁻¹ and then cultivated out (100-200 μl/100 mm-plate) on minimal medium such as KK2 (Fey, P., Kowal, A. S., Gaudet, P., Pilcher, K. E. & Chisholm, R. L. Protocols for growth and development of Dictyostelium discoideum. Nat. Protocols 2, 1307-1316 (2007); www.dictybase.org. Protocols for Dictyostelium discoideum development. http://dictybase.org/techniques/media/dicty development.html (2010)) or MES agar supplemented with CaCl₂ and MgCl₂. Cellulose stalks formed after 16 hours and were cultured up to 3 days and examined at regular intervals.

The presence of the gene and the protein in the host cell was detected by PCR and Western blot.

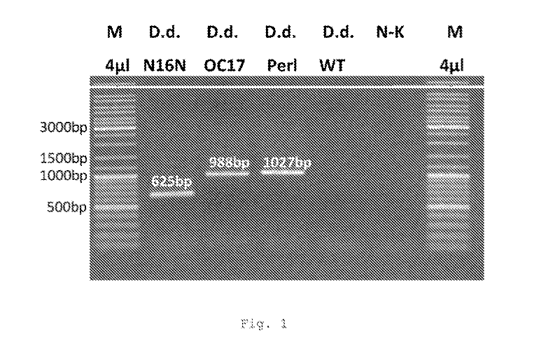

FIG. 1 depicts detection of the genes in a clone by Colony PCR.

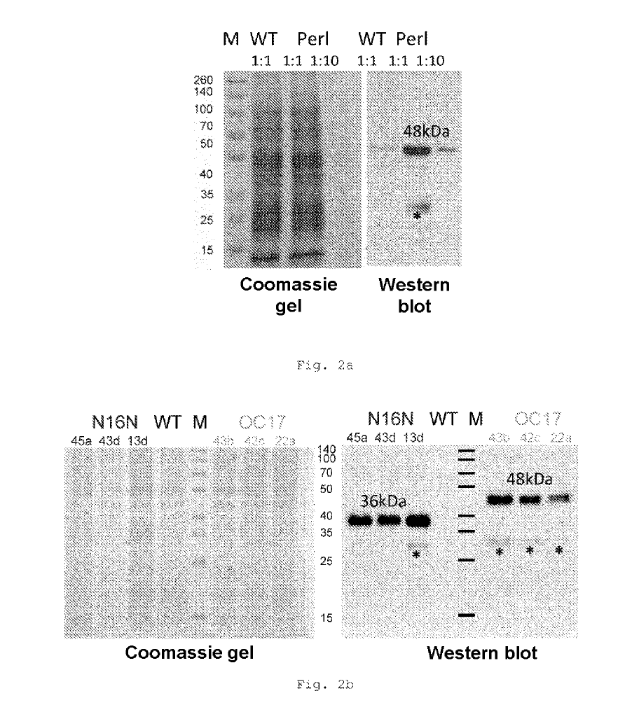

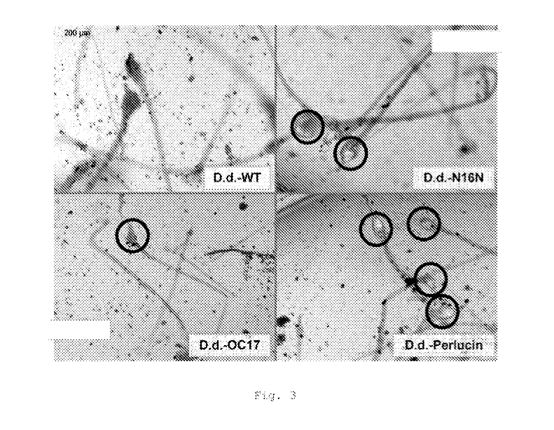

FIGS. 2a and 2b depict detection of the proteins by Coomassie gels and Western blots. An anti-GFP antibody was used as primary antibody for the Western blot.

### Stereomicroscopy:

A Leica M165C (Leica, Germany) was used for stereomicroscopy.

### Light Microscopy and Fluorescence Microscopy

The cell lines were observed using an inverted optical microscope (Cell Observer Z1 with arc lamp and Colibri LED fluorescence excitation).

Confocal laser scanning microscopy, Leica DMR, GFP excitation was effected by an argon laser at 488 nm. GFP emission was detected between 507-512 nm.

### Birefringence Microscopy (LC PolScope):

The birefringence of samples was measured using an LC PolScope with the CRI imaging system, in connection with a Zeiss Observer Z1. The background was measured using measurements without samples (R. Oldenbourg, G. Mei, Journal of Microscopy 1995, 180, 140. R. Oldenbourg, in Live Cell Imaging: A Laboratory Manual, (Eds: R. D. Goldman, D. L. Spector), Cold Spring Harbor Laboratory Press, New York 2004, 205).

### Environmental Scanning Electron Microscopy (ESEM)

The measurements were carried out using a Quanta FEI 400F (FEI, Netherlands) scanning electron microscope with variable pressure (VP-SEM), either under ambient conditions or under low vacuum. The samples were measured without coating.

Raman spectra were recorded using a confocal Raman spectrometer (Aramis Labram) with a cooled detector. The samples were excited by a 785 nm laser and measured using 50× and 100× lenses, a grating of 600 over a range from 100 to 2000 cm⁻¹.

### Location of the Protein

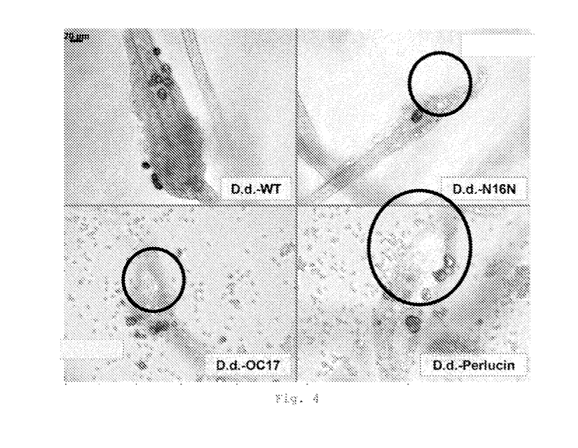

In accordance with the signal sequence used, the expressed protein is located primarily in the heads of the slime mold. This can clearly be seen in FIG. 3 from the GFP fluorescence (black circles in FIG. 3; WT: wild type).

### Precipitation of Calcium Carbonate

For precipitation of calcium carbonate with Dictyostelium clones, 500 μl of CaCl₂ solution (20 mM, sterile filtered) were applied to the Dictyostelium cellulose products and placed in a desiccator. A glass beaker containing 5 g of ammonium carbonate and covered with an aluminum foil with three holes (approx. 5 mm in diameter) was in the same desiccator. The samples were incubated at 22° C. for up to 3 days. Diffusion of ammonium carbonate vapor, causes calcium carbonate to crystallize in this solution.

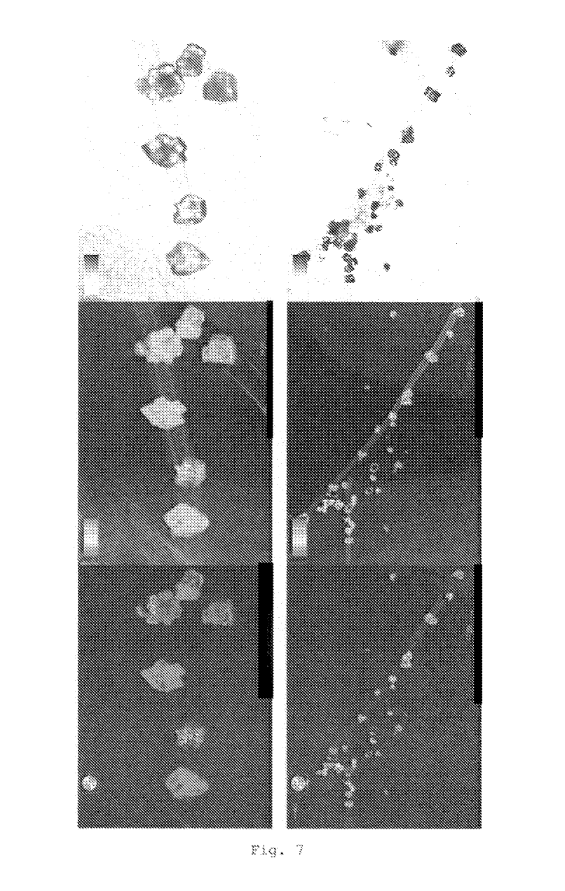

FIG. 6 depicts the regular crystals of 30-45 μm in diameter obtained in the blank sample.

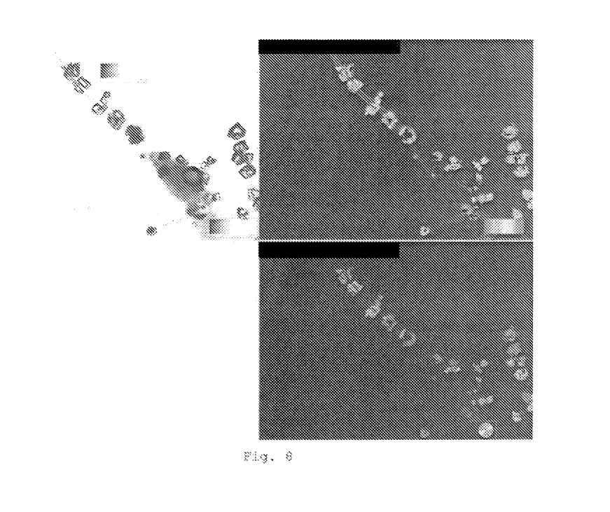

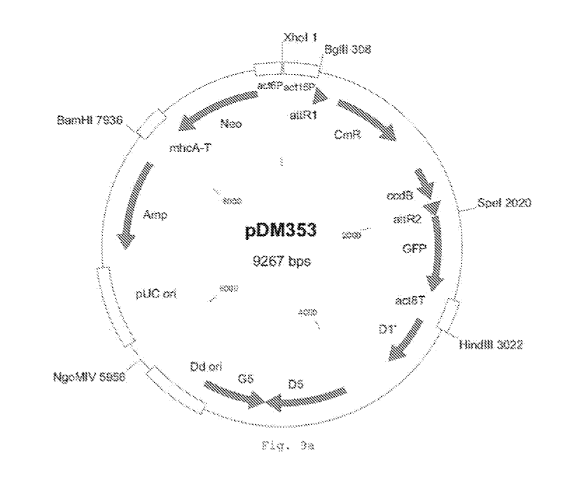

FIG. 7 depicts the crystals obtained with the wild type. FIG. 8 comprises the crystals obtained with the clone. A distinct layered structure of the crystals of the clone, which is not found with the wild type, is visible in particular in the images depicting the birefringence of the crystals.

An alternative protocol (according to Wheeler et al. Science 1981, 212, 1397-1398) used CaCl₂ and NaHCO₃ in 2 mM Glygly at pH 8.5.

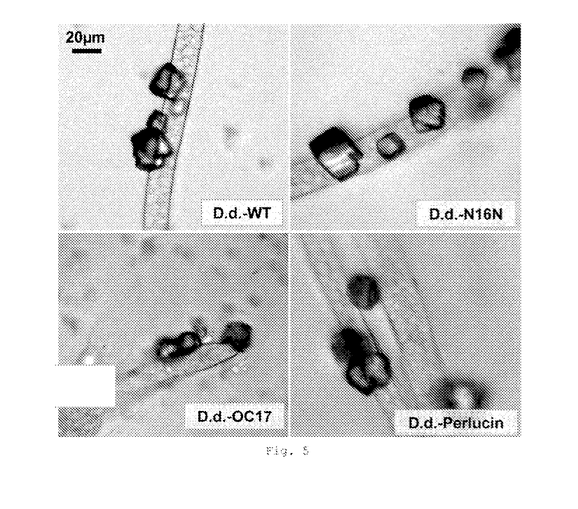

FIG. 4 depicts light microscopy images of the crystals obtained. These images reveal that the crystals on the slime mold and in the exterior space are clearly different.

Another experiment for detecting the function is based on harvesting the Dictyostelium cellulose products, removing the water contained therein by freeze drying (GFP fluorescence is retained which shows that the method is gentle enough for preserving the protein with biomineralizing action in the composite material), and letting said products soak in Ca- or carbonate-containing solutions. Precipitates obtained in this way in the cellulose composite differ between wild type and clone in the optical analysis by means of LC-PolScope.

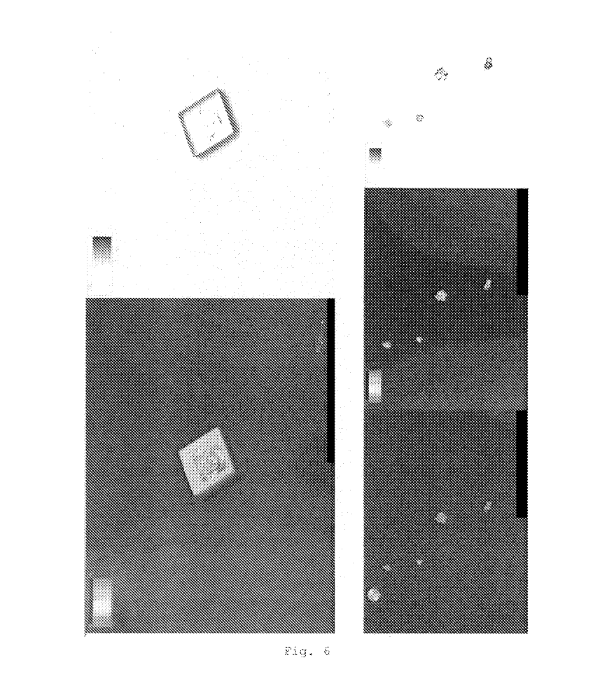

The concomitant fluorescence image indicates that the crystals obtained also fluoresce, indicating that the expressed protein is also present in the crystals and therefore is involved in the biomineralizing process (FIG. 5).

### OTHER EXPERIMENTS

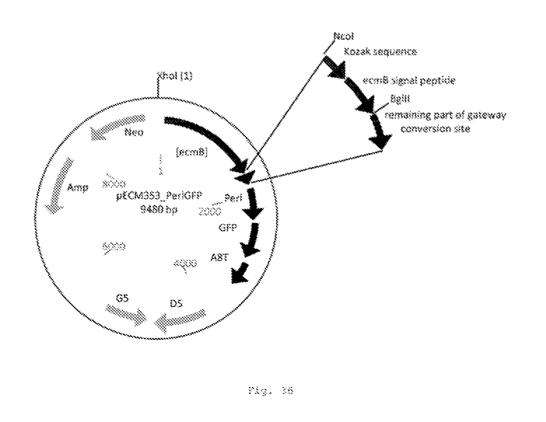

The methods described were used for preparing modified pDM353 vectors with n16N (Seq. ID No. 78), OC-17 (Seq. ID NO. 79) and perlucin (Seq. ID No. 80). The sequences were integrated as described into the pECM353 vectors. The sequences themselves and the flanking sequences were sequenced. Seq. ID 81 depicts the flanking sequence upstream of the protein (5′), with NcoI restriction cleavage site, Kozak sequence, start codon, ecmB signal peptide, BglII restriction cleavage site, and the start of the attL sequence. Seq. ID 82 depicts the flanking sequence in the 3′ direction. FIG. 36 depicts a diagrammatic representation of the vector containing perlucin (pECM353_PerlGFP).

These vectors were cloned into D. discoideum AX3orf⁺ cells. The cell lines obtained are referred to as AX3_n16NGFP, AX3_OC17GFP and AX3_PerlGFP hereinbelow.

Owing to the signal peptide, the fusion protein was expected to be located primarily in the extracellular matrix of cells of the stalk.

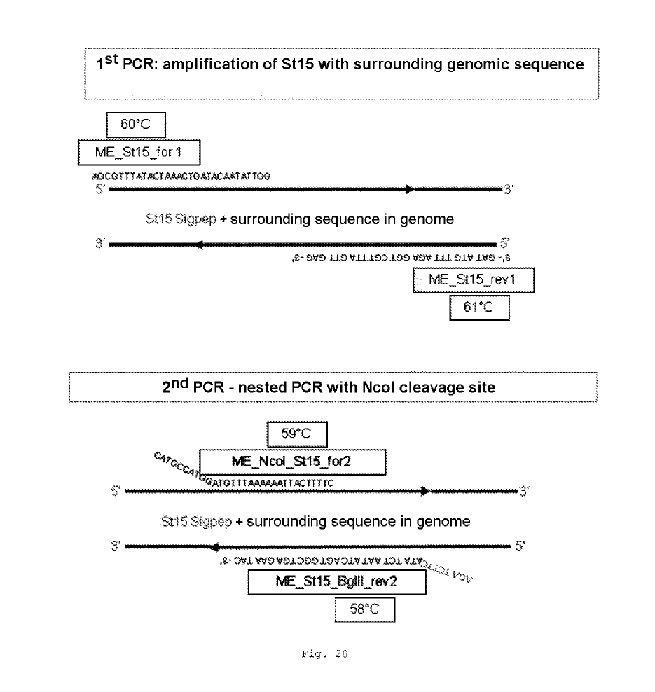

FIG. 22 depicts light microscopy and fluorescence microscopy images of the stalk (a, b) and of the central region (c-j, region 3 according to the arrows in a) and of the basal stalk region (k-r, region 1 according to the arrows in a). The images show Ax3-Orf+ cell lines containing n16NGFP (a, b, c, d, k, l), OC17GFP (e, f, m, n), PerlGFP (g, h, o, p) and the untransformed reference cell lines Ax3-Orf+ (i, j, q, r). All images were recorded under the same conditions. The scale bars represent 100 μm (a, b) 100 μm and 20 μm (c-r).

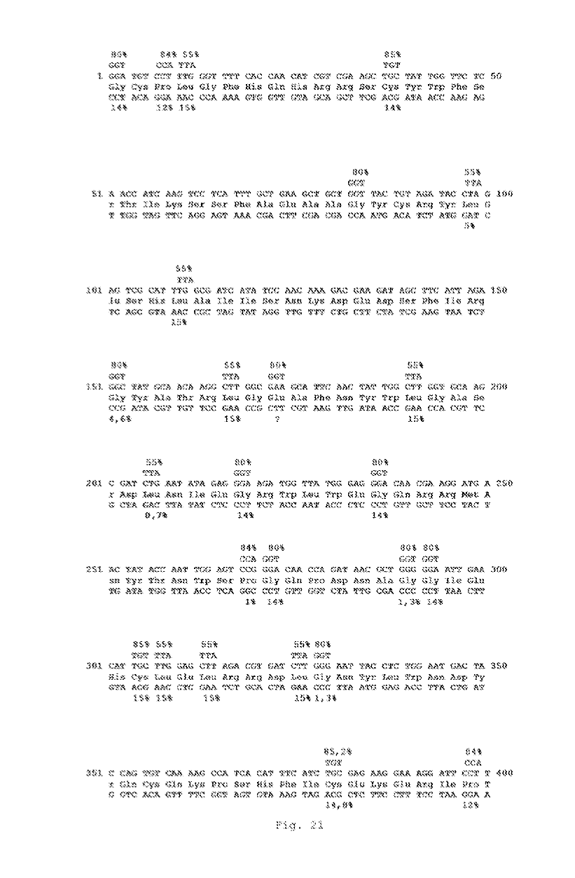

There were only small morphological differences visible between the modified cell lines and the unmodified cell line. AX3_n16NGFP sometimes had a rougher surface in the lower region. In rare cases, AX3_OC17GFP showed a narrower stalk or small widenings. FIG. 23 depicts images of the entire mold (a-d) and of the basal disk (region 1, e-h) of the Ax3-Orf+ cell line (a, e), AX3_n16NGFP (b, f), AX3_OC17GFP (c, g), and AX3_PerlGFP (d, h). The arrows indicate rare morphologies of the cell lines (scale: 200 μm (a-d) and 100 μm (e-h)).

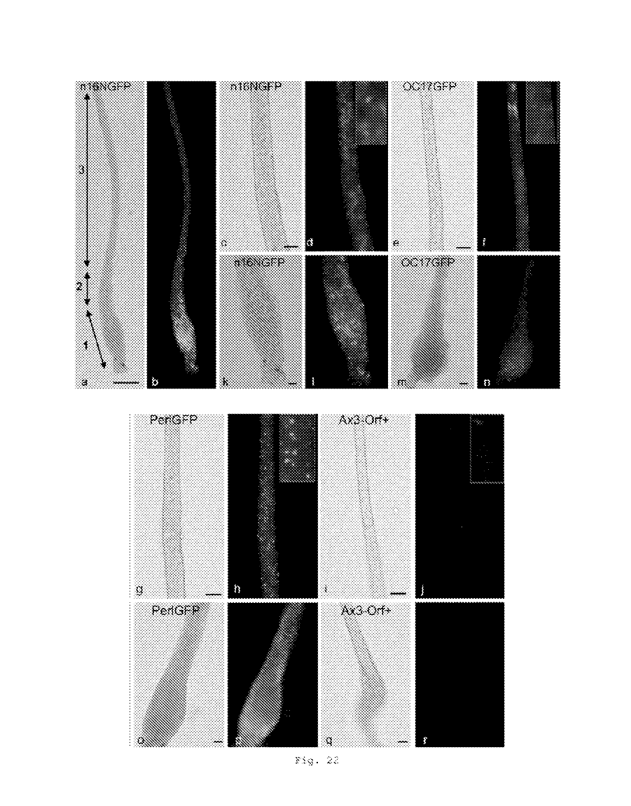

FIG. 24 depicts a quantitative fluorescence analysis. The figure shows statistical averages of fluorescence in the lower region of the stalk (a) and in the central region of the stalk (b). For this purpose, the gray value of the image was determined (Axiovision software, Zeiss). The average was determined from at least 21 samples of at least 3 experiments. The error bars indicate the standard deviation. The samples are the reference cell line (Ax3-Orf+), and AX3_n16NGFP (n16N), AX3_OC17GFP (OC17), and AX3_PerlGFP (Perl). The asterisks indicate cell lines with a significant difference (Student T's distribution; p≦0.05). The data reveal that the modified cells exhibit a higher fluorescence than the unmodified cells.

As FIG. 22 also shows, the fluorescence is concentrated primarily in the basal disk (region 1). In the case of perlucin, fluorescence distribution is somewhat more diffuse. Increased fluorescence was likewise measured in the modified cell lines at the upper and lower end of the spore head. This is typical for the ecmB promoter.

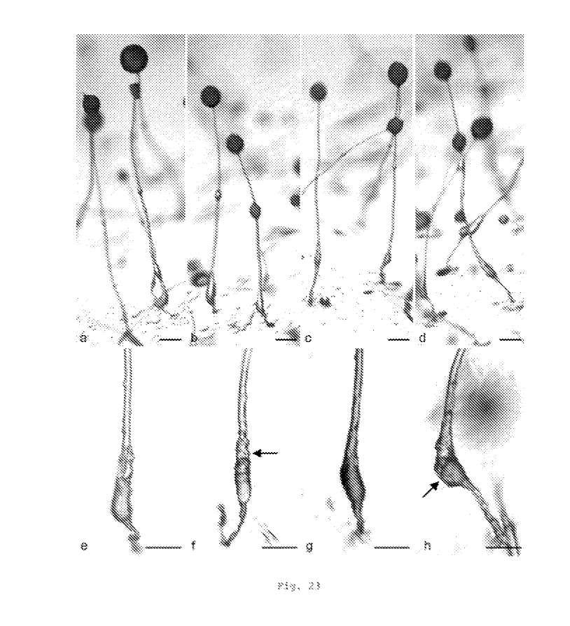

The GFP fluorescence signals also correspond to the crystals formed later. FIG. 25 depicts a superimposition of a light microscopy image and a fluorescence microscopy image (top: superimposition; bottom: fluorescence image) of region 1 of AX3_n16NGFP (scale 10 μm). The arrows indicate the crystals. This shows that the modified fusion proteins must also be present in the extracellular matrix.

The experiments indicate that the modified proteins are expressed in the modified cell lines, and that expression is under the control of the ecmB promoter. The proteins are also incorporated into the extracellular matrix of the cells. Some of the modified proteins were also detected in the medium.

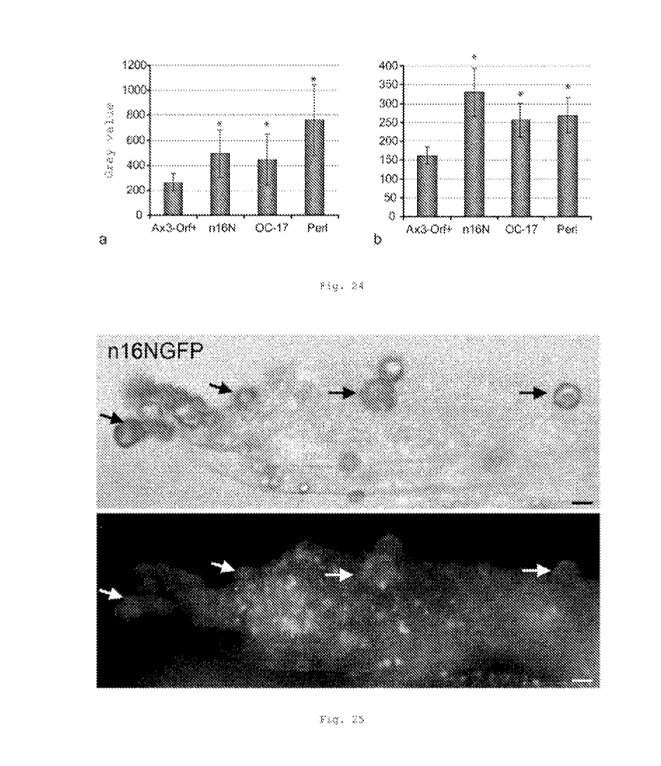

The proteins were also detected by Western blots using a monoclonal anti-GFP antibody. FIG. 26 depicts Western blots of protein extracts from modified D. discoideum Ax3-Orf+ transformed with the vectors pECM353_PerlGFP (1), pECM353_OC17GFP (2), pECM353_n16NGFP (3), and the unmodified cell line Ax3-Orf+ as control (4). The marker bands are indicated in kDa. The strong protein bands correspond to the complete heterologous proteins after removal of the signal peptide.

### Mineralization:

When cells were cultured in MES medium, no mineral phases were found with AX3_n16NGFP, AX3_OC17GFP and AX3_PerlGFP.

Mineralization was investigated by incubating either MES agar plates with cultured cell lines with 0.5 g of solid (NH₄)₂CO₃ in a sealed chamber at 22° C.+/−1° C. for 16 to 48 hours. For some experiments, 20 mM or 50 mM CaCl₂ were added to the agar plates.

Mineralization by carbonate vapor diffusion proved to be particularly advantageous. For this purpose, the stalks of Distyostelium discoideum cell lines aged 2-3 days were collected using tweezers, and the spore heads were removed. The stalks were immersed in 25 μm of 10 mM CaCl₂ (pH 6.0) prepipetted on microscopy slides. The glass plates were covered with perfluorinated aluminum foil and incubated at room temperature in a closed space at room temperature for 12 hours to 2 days. In order to maintain a humid atmosphere in the space, said space additionally contained 0.5 g of (NH₄)₂CO₃ in 10 ml of distilled water. During mineralization, the pH of the sample increased to 8.7+/−0.2.

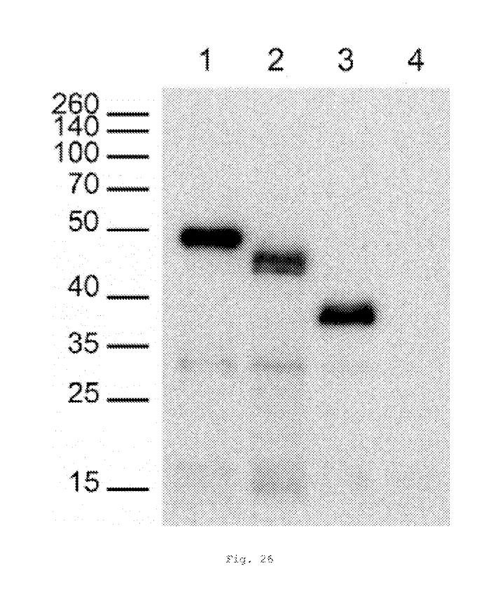

This method produced different morphologies of crystals on the stalk surface. FIG. 27 depicts images of these crystals. a) depicts the crystals on an AX3-Orf+ stalk. b) depicts crystals without slime molds in the precursor solution. The arrows in a) indicate the basal region (region 1) and the central stalk region (region 3). The bottom images depict magnified sections from region 1 of Ax3-Orf+(c) and AX3_n16NGFP (d), AX3_OC17GFP (e), and AX3_PerlGFP (f) as overlay of light microscopy and fluorescence microscopy images (scale: (a, b) 100 μm, (c-f) 20 μm).

The crystals of regions 1 and 3 were analyzed. The crystals in region 1 were smaller and more spherical than the crystals in region 3.

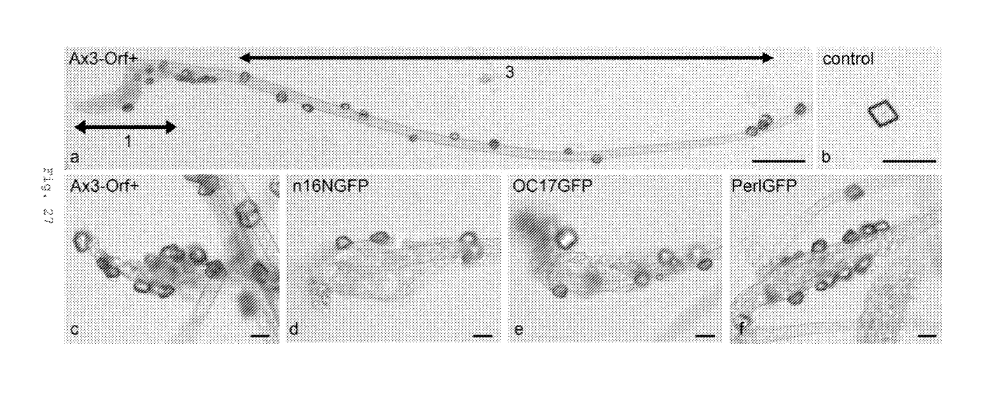

FIG. 28 depicts SEM images of calcium carbonate crystals obtained on the different cell lines by carbonate vapor. a) depicts a rhombic crystal from the solution. Crystals of region 1 are depicted in (b-f), of region 3 in (g-h). The crystals were classified into individual rhombohedrons (b, g), stepped rhombohedrons (c, h), polycrystals with differently oriented subunits (d, i), crystals with round edges (e, j) and round precipitations (f, k, l). Scale: (a) 20 μm, (b-l, r) 10 μm, (j) 15 μm, (k) 5 μm.

The precipitations adhere to the stalks so strongly that it was possible to transfer them to filter membranes and remove the solvent by suction. The samples were then washed twice with distilled water (100 μl).

The crystals were classified into different morphologies on the basis of the SEM images. Images of different assays were used. Classification was carried out by two persons independently of one another.

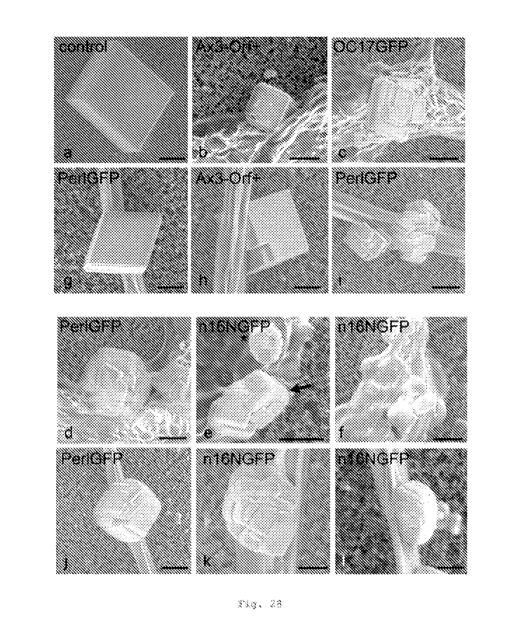

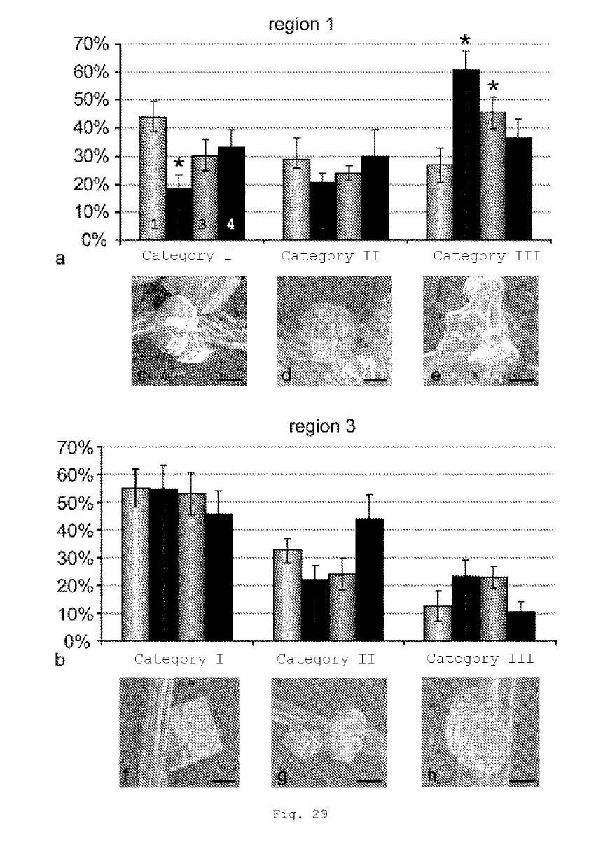

FIG. 29 depicts the relative distribution of morphologies in region 1 (a; base) and region 3 (b; stalk) of Ax3-Orf+ control cell line (1^{st} column), and of AX3_n16NGFP (2^{nd} column), AX3_OC17GFP (3^{rd} column), and AX3_PerlGFP (4^{th} column) cell lines. The bottom images depict representative morphologies of region 1 (c-e) and region 3 (f-h). The crystals were classified into stepped rhombohedrons (category I; c, f), polycrystalline rhombohedrons with differently oriented subunits (category II, d, g), and crystals with a plump appearance (category III, e, h). Percentages were calculated from 6 different assays. Significant differences according to Student's T test (p 0.05) compared to the Ax3-Orf+ control are indicated by an asterisk. Scale (c, h) 5 μm (d-g) 10 μm.

It turns out that the variety of crystal forms in the modified cell lines is markedly higher than in the unmodified cell lines. The proteins in the extracellular matrix therefore influence formation of the crystals.

The crystals were also examined by LC PolScope. This technique allows easier screening of the crystal forms formed.

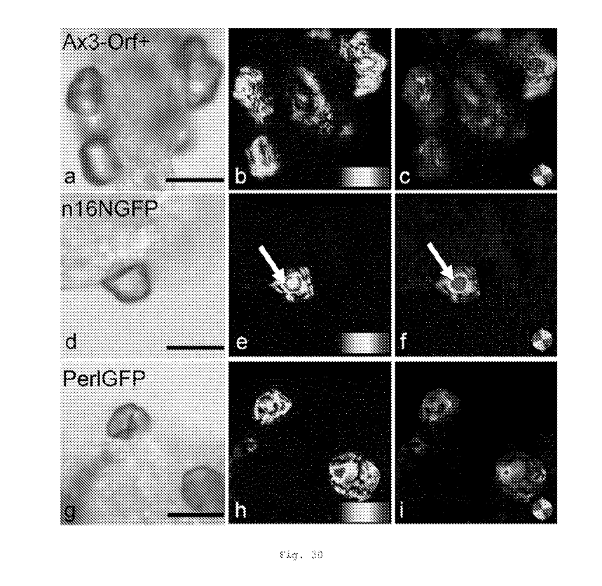

FIG. 30 depicts images of crystals in the basal region of Ax3-Orf+(a-c), and of AX3_n16NGFP (d-f) and AX3_PerlGFP (g-i) as superimposition of light microscopy and fluorescence microscopy images and LC PolScope images analyzed in “retardance mode” (b, e, h) and in “orientation mode” (c, f, i). Retardance scale (black to red), (b) 0-272 nm, (e) 0-268 nm, and (h) 0-270 nm. Orientation of the slow optical axis in (c, f, i), red (right hand side in the spectrum) 0°/180°, light blue 90°/270°. Scale: 20 μm.

It is clearly visible that crystals were obtained for AX3_n16NGFP which have a delimited inner sphere (arrows). The circle in image e) is red.

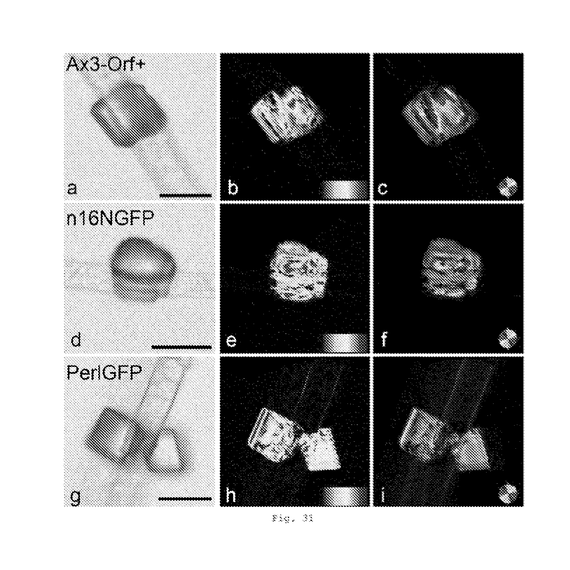

FIG. 31 depicts images of crystals of the stalk region of Ax3-Orf⁺ (a-c), and of AX3_n16NGFP (d-f) and AX3_PerlGFP (g-l) as superimposition of light microscopy and fluorescence microscopy images and LC PolScope images analyzed in “retardance mode” (b, e, h) and in “orientation mode” (c, f, i, l). Retardance scale (black to red), (b) 0-269 nm, (e) 0-232 nm, and (h) 0-255 nm. Orientation of the slow optical axis in (c, f, i), red (right hand side in the spectrum) 0°/180°, light blue 90°/270°. Scale: 20 μm.

This too shows that the extracellular matrix of AX3_n16NGFP promotes the construction of multilayered crystal forms.

In addition, the interface between the crystals and the extracellular matrix was investigated.

For this purpose, crystals were examined that had formed around the stalk.

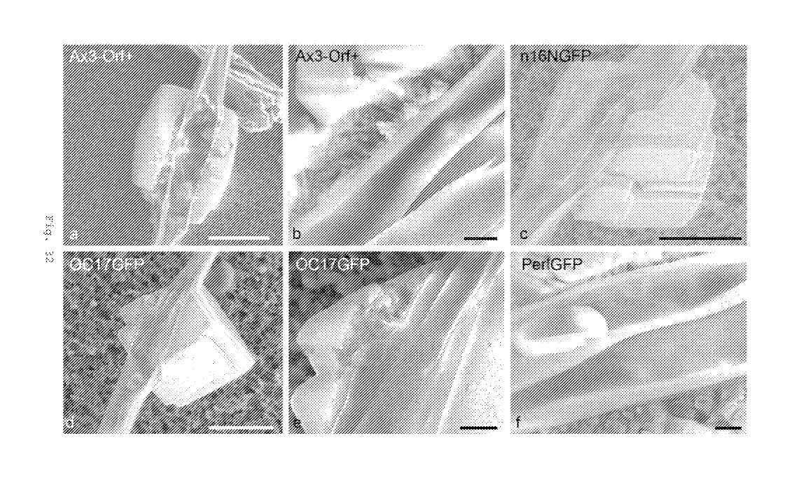

FIG. 32 depicts low vacuum SEM images of stalk-surrounding crystals. Non-intercalating crystal-organic interfaces were found in all assays. Examples of such interfaces are shown in the figures for Ax3-Orf+ (a, b) and AX3_n16NGFP (c). Intercalating interfaces were found only in AX3_OC17GFP (d, e) and AX3_PerlGFP (f). Scale: 20 μm (a), 10 μm (c, d), 5 μm (b), and 2 μm (e, f).

The crystals adhere very tightly to the stalk and also adapt their interface to the shape of the stalk.

However, the interface for AX3_OC17GFP and AX3_PerlGFP (FIG. 32 e, f) is markedly less defined than in unmodified strains (FIG. 32 a, b).

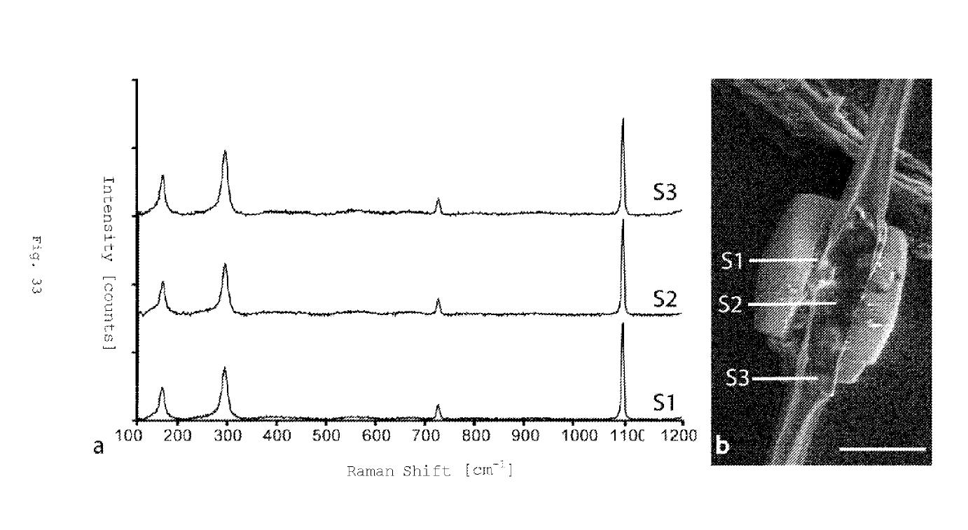

FIG. 33 depicts correlated VP-SEM and Raman microscopy of Ax3-Orf⁺. Raman spectra (a) were recorded at some sites (S1 to S3) where the crystal encloses the stalk. The positions are indicated in the SEM image (b). Scale: 20 μm.

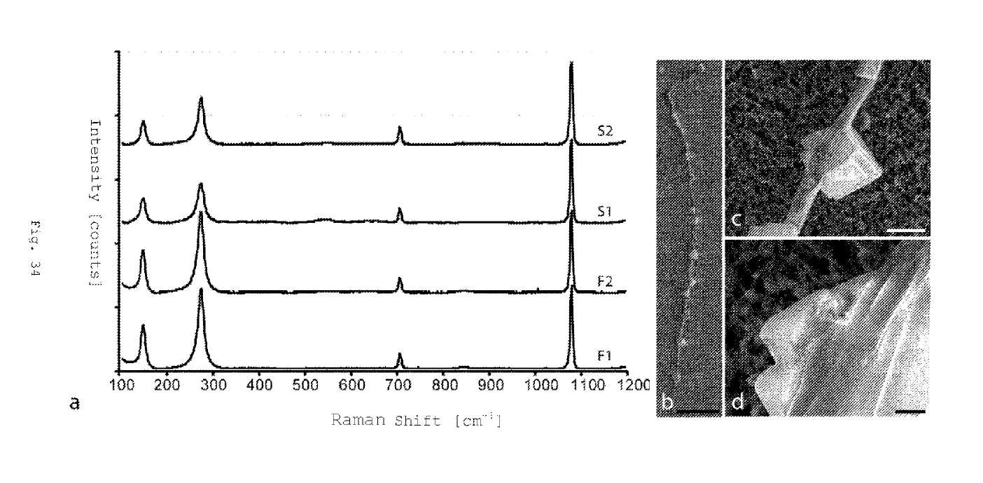

FIG. 34 depicts correlated VP-SEM and Raman microscopy of AX3_OC17GFP. Raman spectra (a) were recorded of crystals in region 1 (F1, F2) and region 3 (S1, S2); higher resolution image of the crystal from region 3 (c) and stalk-crystal interface (d); (d) shows an intercalation of the inner crystal-organic interface; scale (b) 100 μm, (c) 10 μm, (d) 2 μm.

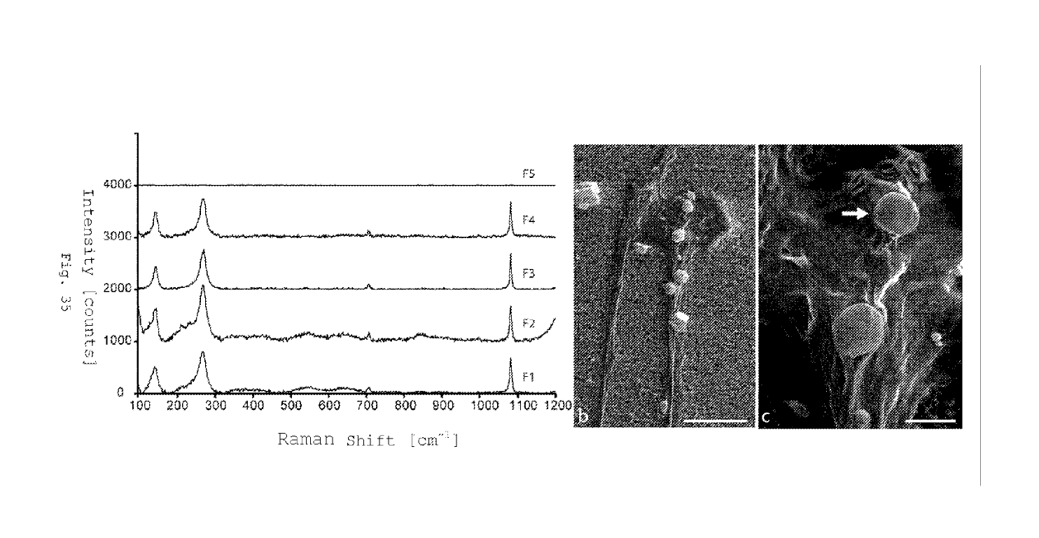

FIG. 35 depicts correlated VP-SEM and Raman microscopy of a crystal in region 1 of AX3_n16NGFP in an overview (b) and in detail (arrow c). Raman spectra (a) were in confocal mode with 5 steps starting in the nitrocellulose filter (F1), through the crystal (F2 to F4) and ending slightly above the crystal (indicated by the arrow in c)). All spectra were normalized to the height of the band at 1086 cm⁻¹. Scale: (b) 50 μm, (c) 10 μm.

Raman spectra did not show any fundamental differences between the crystals obtained. All crystals had calcite. This was also confirmed by XRD.

The Raman signature of the extracellular matrix (FIGS. 35 F1 and F2) differs from that of calcite (FIGS. 35 F3 and F4). With focusing above the crystal, a Raman signal can no longer be measured (FIG. 35 F5).

TABLE 1

| | | Direct | |
|---|---|---|---|
| | | contact | |
| Amino acid | Protein | between amino | Water-mediated |
| motif | example | acids (A-A) | contact |
| Asp (Poly-D) | RP-1 | −/− | +++++ |
| | Caspartin | (D-K) | |
| | MSP-1 | (D-R) | |
| | Aspein | | |
| | Asp-rich | | |
| | family | | |
| Asn (N), Gly | Caspartin | Moderate | Moderate |
| (G) | | | |
| GDN/GNN | Nacrein | D-N | (charged/polar |
| | | | amino acid) |
| Poly-G | MSI-60 | G-G | |
| | MSI-31 | | |
| G-rich | MSI-7 | | |
| N-rich | MSP-1 | | |
| | LustrinA | | |
| | AP-24 | | |
| Ala (poly-A) | MSI-60 | +++++ | Moderate |
| A-rich | AP-24 | Hydrophobic | No difference |
| | | amino acid | |
| Ser (S-rich) | MSI-31 | + (only Cys) | +++++ |
| | | S-C | S-P |
| Ser/Pro | Mucoperlin | −/− | +++++ |
| (SP-rich) | Lustrin A | No direct P-X | Strongly |
| P-rich | | (X = charged | preferred |
| | | amino acid) | P-S |
| Ser/Gly | MSP-1 | Moderate | Moderate |
| (GS-repeats) | Lustrin | S-C | S-P |
| Thr (T-rich) | AP-24 | Moderate | Moderate |
| Val (V-rich) | MSI-31 | +++++ | Moderate |
| | | Hydrophobic | No difference |
| | | amino acid | |
| His (H-rich) | EP fluid | +++++ | +++++ |
| | protein | Hydrophobic | Charged/polar |
| | (Mytilus | amino acid | amino acids |
| | edulis) | | |
| Cys (C-rich) | LustrinA | ++ | ++++ |
| | AP-4 | C-H, C-S, C-M | Preferred |
| | Perlustrin | Hydrophobic | No difference |
| | | amino acid | |

TABLE 2

| | | | | Function (for recognized enzymes |
|---|---|---|---|---|
| Kingdom | | | | with EC number) |
| Phylum | | | SwissProt Number/ | Domains/patterns in the primary |
| Species/Species | Mineral | Name | EC number | structure domains |
| Bacteria | | | | |
| α-Proteobacteria | | Nitrogenase reductase | Q1HI63/EC 1.18.6.1 | Iron-binding |
| Magnetotactic bacteria | | Presumably type I | Q3BK95/EC 3.6.3.— | Transporter family |
| | | secretion system | | |
| | | ATPase HlyB | | |
| Magnetospirillum | | MamU | Q3BK99/Q6NE47/EC | Diacylglycerol kinase activity |
| gryphiswaldense | | | 2.7.1.107? | |
| | | MamW | Q5D4Z7/ | Presumably sulfate permease |
| | | MamE | Q6NE61/EC 3.4.21.— | Presumably iron-binding/serine |
| | | | | protease activity |
| | | MamO | Q93DZ1/EC 3.4.21.— | Serine-type endopeptidase |
| | | | | activity |
| | | Mms16 | Q6NE80/EC 3.6.—.—? | GTPase activity |
| | | MamJ | Q3BKB2/Q6NE60 | Acidic, Glu-rich, repeats |
| | | MamH | Q3BKB5/Q6NE63 | Ala-Val-Leu-rich |
| | | Hemerythrin- | Q3BKC0/Q3BKC1/Q6NE67 | Iron-binding |
| | | like proteins | | |
| | | Ferric iron-binding | Q3BKC4 | Iron-binding |
| | | protein | | |
| | | Mms6 | Q3BKD2/Q6NE76 | Hydrophobic, Ala-Gly-Val-rich |
| | | MamD | Q93DY2/Q6NE73 | Ala-Gly-Thr-rich |
| | | MamG | Q6NE75 | Ala-Gly-leu-rich |
| | | TPR protein | Q3BKD5 | Hydrophobic, binding |
| | | (tetratricopeptide) | | |
| | Fe₃O₄ | MamA | Q93DY9 | TPR-like |
| | (magnetite) | Acidic protein | Q3BKD7 | Pentapeptide repeats |
| | | MM22 | Q6N0B5 | Ala-Val-rich |
| | | MamT | Q93DY4/Q6NE48 | 2 presumably cytochrome c heme- |
| | | | | binding sites |
| | | MamS | Q6NE49 | Hydrophobic, Ala-Gly-Leu-rich |
| | | MamB | Q93DY6/Q6NE50 | Ala-Val-rich |
| | | MamM | Q6NE57 | Val-rich, similar to MamB |
| | | MamR | Q6NE51 | Leu-rich |
| | | MamN | Q6NE56 | Ala-Gly-Leu-rich |
| | | MamL | Q6NE58 | Basic, Val-Gly-Leu-rich |
| | | MamK | Q6NE59 | |
| | | MamI | Q6NE62 | Ala-Leu-rich |
| | | MamC | Q93DY1/Q6NE72 | Ala-Gly-Leu-rich |
| | | MamF | Q6NE74 | Leu-rich, basic |
| | | MamQ | Q93DY8 | Hydrophobic |
| | | MamP | Q93DZ0 | Val-Ala-Gly-rich |
| | | Presumably IdiA | Q6NE71 | Ala-rich |
| | | Bacterioferritin | Q6NE81/Q6NE82 | Ferritin-like |
| β-Proteobacteria | MnO₂ | Phosphoglycerate | P71430/EC 5.4.2.1 | |
| | | mutase | | |
| Mn/Fe oxidic bacteria | (+ Fe-ox) | MofA | P71431/EC 1.—.—.— | Copper ion-binding, oxidase |
| Leptothrix discophora | | MofB | P71432/EC 5.2.1.8 | FKBP-type PPIase (FKBP-type |
| | | | | peptidylprolyl cis-trans |
| | | | | isomerase) |
| | | MofC | Q9X760 | |
| | | Secreted protein | Q48532 | Transporter activity |
| Eucarya | | | | |
| Bacillario phyta | SiO₂ | RubisCO | P24673/P24683/EC | Carboxylation of D-ribulose 1,5- |
| (Diatoms) | | | 4.1.1.39 | bisphosphate, oxidation of |
| | | | | pentose |
| Cylindrotheca | | Silaffin-1 | Q9SE35 | Acidic N-ter., Ser-Lys-Gly-rich |
| fusiformis | | | | repeats, P-Ser, OH-Lys, poly- |
| | | | | amine-Lys |
| | | HEP200 prot. | O22015 | Pro-rich, 5 PSCD repeats, acidic |
| | | | | protein |
| | | HEPB | O22016 | Pro-rich, 4 PSCD repeats, acidic |
| | | | | protein |
| | | HEPC | O22017 | Short Pro-rich, 3 PSCD repeats, |
| | | | | acidic protein |
| | | P75K | Q39494 | Pro-Asp-Ser-Gly-rich, 5 acidic |
| | | | | Cys-rich (ACR) repeats |
| | | a1-3-frustulins, | Q39495/Q39496 | Acidic Cys-rich (ACR) repeats, |
| | | e-frustulin | | Pro-rich, Poly-Gly |
| | | SIT1-5 | O81199 to O81203 | Leu-rich, Cys pattern |
| Thalassiosira | SiO₂ | Glutamate | EC 2.3.1.1 | |
| pseudonana | | acetyltransferase | | |
| | | Arginase | EC 3.5.3.1 | |
| | | Ornithine | EC 2.1.3.3 | |
| | | carbamoyltransferase | | |
| | | Ornithine | EC 4.1.1.17 | |
| | | decarboxylase | | |
| | | SAM-decarboxylase | EC 4.1.1.50 | |
| | | Trypsin-like | EC 3.4.21.— | Serine endopeptidase |
| | | Serine protease | | |
| | | Serine/Threonin | EC 2.7.11.— | Phosphorylation of Ser/Thr |
| | | Protein kinase | | |
| | | a-kinases | EC 2.7.—.— | Phosphorylation |
| | | Ubiquitin ligase | EC 6.3.2.— | |
| | | Serine protease | | |
| | | inhibitor, Kazal-like | | |
| | | TPR protein | | Tetratricopeptide |
| | | HSP70-TPR-like | | Tetratricopeptide repeats |
| | | SIT1-3 | Q0QVM6-8 | Leu-rich |
| | | Sillafins 1-3 | Q5Y2C0-2 | Ser-Ala-Lys-rich (Sil3), |
| | | | | Ser-Pro-Thr-rich (Sil1-2) |
| Haptophy-phy- | CaCO₃ | g-Carbonic anhydrase | Q0ZB85/EC | Presumably acyltransferase |
| Ta (Coccolitophores) | | | 4.2.1.1/EC 2.3.1.—? | activity |
| Emiliania huxleyi | (Calcite) | d-Carbonic anhydrase | Q0ZB86/EC 4.2.1.1? | |
| | | (d-EhCA1) | | |
| | | Phosphate permease | Q8GZT5/ | |
| | | Alkaline phosphatase | Q09HD2/EC 3.1.3.1 | |
| | | Arachidonate 15- | Q0MYU5/EC 1.13.11.33 | |
| | | lipoxygenase, 2^{nd} type | | |
| | | RubisCO | Q4G3F4/EC 4.1.1.39 | Carboxylation of D-ribulose 1,5- |
| | | | | bisphosphate |
| | | L-3-phosphoserine | Q0MYU8/EC 3.1.3.3 | |
| | | phosphatase | | |
| | | Cysteine protease | Q0MYX5/EC 3.4.22.— | Presumably cysteine type |
| | | | | endopeptidase activity |
| | | Esterase | Q4A2B6/EC 3.1.—.— | Hydroxylase activity |
| | | IMP dehydrogenase/GMP | EC 1.1.1.205? | Synthesis of GMP? |
| | | reductase | | |
| | | Ser/Thr kinase | Q0MYX0/EC 2.7.11.—? | Presumably protein Ser/Thr |
| | | | | phosphorylation |
| | | Presumably Ca-binding | Q0MYW8 | Ala-Glu-Pro-rich, presumably Ca- |
| | | prot. | | binding |
| | | GPA | Q9ZTY0 | Ala-Glu-Pro-rich, EF hand |
| | | Presumably SNAP | Q0MYX6 | Ala-Lys-rich |
| | | Presumably | | Ala-Arg-Ser-rich |
| | | RabGAP/TBC-containing | Q0MYX4 | |
| | | protein | | |
| | | Presumably basic | | Ala-Gly-rich |
| | | protein | | |
| | | Presumably | | Glycosylated |
| | | glycoproteins | | |
| Metazoans | | | | |
| Porifera (Sponges) | SiO₂ | Silicatein a | Q2MEV3/EC 3.4.22.— | Cysteine type endopeptidase |
| | | | | activity (C1 peptidase) |
| Suberites domuncula | | Silicatein b | Q50IU7/EC 3.4.22.— | Cysteine type endopeptidase |
| Spicules | | | | activity (C1 peptidase) |
| | | Cathepsin X/O | Q6A1H9/EC 3.4.22.— | Cysteine type endopeptidase |
| | | (cat-X/O) | | activity (C1 peptidase) |
| | | Cathepsin L (cat-L) | Q6A1IO/EC 3.4.22.— | Cysteine type endopeptidase |
| | | | | activity (C1 peptidase) |
| | | Cathepsin H (cat-H) | Q6A1I1/EC 3.4.22.— | Cysteine type endopeptidase |
| | | | | activity (C1 peptidase) |
| | | Cathepsin B (cat-B) | Q6A1I2/EC 3.4.22.— | Cysteine type endopeptidase |
| | | | | activity (C1 peptidase) |
| | | Arginine kinase | Q4W3A2/EC 2.7.3.3 | Presumably phosphorylates |
| | | | | Arg residues |
| | | Protein tyrosine | Q50IU8/EC 2.7.10.— | Phosphorylates Tyr residues in |
| | | kinase | | proteins |
| | | Silicase | No number (EC 4.2.1.1) | Carbonic anhydrase-like, 3 Zn- |
| | | | | binding His |
| | | Isocitrate | No number (EC 1.1.1.41) | |
| | | dehydrogenase (SDIDH) | | |
| | | Galectin | Q1MSI8 | Galactose-binding, hydrophobic |
| | | | | N terminus |
| | | Mannose-binding | No number | Mannose-binding |
| | | Lectin (MBL) | | |
| | | Silicatein protein | Q4A3I9 | Ser-Thr-Val-rich, tandem repeats |
| | | Selenoprotein M | Q4A3I8 | Glu-Leu-rich, presumably a |
| | | | | selenocysteine |
| | | NBCSA | Q684N3 | NBC transporter family |
| | | Collagen | Q9GV99 | Collagen |
| Cnidaria (Corals) | CaCO₃ | Ca²⁺-ATPase (PMCA, p | Q6UUX1/EC 3.6.3.8 | Calcium ion-binding and |
| | | type) | | transport |
| Stylophora pistillata | (Arag.) | RubisCO | AlL0Q2/EC 4.1.1.39 | Carboxylation/oxygenation of |
| | | | | ribulose I,5-bisphosphate |
| | | L type calcium | 097017 | Calcium ion-binding and |
| | | channel a-1 sub. | | transport |
| | | N type calcium | 097128 | Calcium ion-binding and |
| | | channel a-1 sub. | | transport |
| | | P type calcium | 097129 | Calcium ion-binding and |
| | | channel a-1 sub. | | transport |
| L. crassum | CaCO₃ | Carbonic anhydrase | P84537/EC 4.2.1.1 | |
| | | MPL-2 | | |
| L. virgulata | CaCO₃ | Spindel collagen | EC 3.4.24.—? | Collagenase activity |
| | | degrading | enzyme | |
| | | (MMP?) | | |
| G. fascicularis | CaCO₃ | Galaxin | Q8I6S1 | Cys-rich tandem repeats |
| Mollusca (Mollusks) | CaCO₃ | Carbonic anhydrase | EC 4.2.1.1 | |
| Pinctada fucata | (Arag. + | ATP synthase b | Q2HZD8/EC 3.6.3.14 | |
| | Calcite) | subunit | | |
| | | H-ATPase | Q000T7/EC 3.6.3.— | Proton transport |
| | | L type Ca channel | A1Z089 | |
| | | b-subunit | | |
| | | Ser/Thr protein | Q4KTY1/EC 2.7.11.1 | |
| | | kinase H1 (PSKH1) | | |
| | | IKK-like prot. | Q2VU38/EC 2.7.11.10 | Protein serine/threonin kinase |
| | | | | activity |
| | | Glyceraldehyde 3- | Q5DVW2/EC 1.2.1.9 | Glycolysis |
| | | phosphate | | |
| | | dehydrogenase | | |
| | | Alkaline phosphatase | Q17TZ1/EC 3.1.3.1 | Phosphatase activity |
| | | Acid phosphatases | EC 3.1.3.2 | |
| | | Tyrosinase | Q287T6/EC 1.14.18.1 | Presumably monophenol |
| | | | | monooxygenase activity |
| | | Tyrosinase-like | A1IHF0/EC 1.14.18.1 | Cu-binding sites |
| | | protein 1 (Pfty1) | | |
| | | Tyrosinase-like | A1IHF1/EC 1.14.18.1 | Cu-binding sites |
| | | protein 2 (Pfty2) | | |
| | | Astacin-like | Q2VU37/EC 3.4.24.21 | Endopeptidase |
| | | metalloproteinase | | |
| | | (Pf-ALMP) | | |
| | | Nacrein (carbonic | Q27908/EC 4.2.1.1 | 2 CA subdomains + some GXN |
| | | anhydrase) | | repeats |
| | | Prismalin-14 | Q6F4C6 | PIYR repeats, Asp-rich, Gly-Tyr- |
| | | | | rich |
| | | MSI25 | A1IGV6 | Poly-Asp, poly-Ala |
| | | PFP-16 | A1IGV7 | Gly-rich, glycosylated |
| | | MSI-31 | O02401 | Poly-Gly, ESEEDX repeats |
| | | MSI-60 | O02402 | Poly-Ala, poly-Gly, short |
| | | | | Acidic motifs |
| | | PFMG1-12 (11 | Q3YL58-64, Q45TK0-1, | Ca-binding, KAZAL, Ser protease |
| | | proteins) | Q45TJ8-9 | inhibitor |
| | | Pearlin/N14 | Q97048/Q9TVT2/Q9TW98/Q9UAH3 | GN motive |
| | | KRMP1/2/3 | Q1AGV8/9/Q1AGW0 | Lys-rich |
| | | Shematrin1-7 | Q1MW90 to Q1MW96 | Hydrophobic repeats, Arg-Lys- |
| | | | | rich repeats |
| | | Aspein | Q76K52 | Poly-Asp, very acidic |
| | | MSI-7 | Q7YWA5 | Gly-rich |
| | | P10 | No sequence | Asx-Ala-rich |
| | CaCO₃ | Chymotrypsine-like | P35003/EC 3.4.21.— | Ser endopeptidase |
| | | serine proteinase | | |
| Haliotis sp. | (Arag. + | Lustrin A | O44341 | Cys-rich/Pro-rich repeats, |
| | Calcite) | | | GS, basic, protease inhibitor |
| | | AP7 | Q9BP37 | Short hydrophobic/hydrophilic |
| | | | | blocks |
| | | AP24 | Q9BP38 | Short, acidic motifs, N-linked |
| | | | | saccharides |
| | | AP8a-b | No sequence | Asx-Gly-rich |
| | | Perlucin | P82596 | C-type lectin, 2 repeats, N- |
| | | | | linked saccharides |
| | | Perlwapin | P84811 | 3 WAP, Cys-rich |
| | | Perlustrin | P82595 | IGF-BP |
| Crassostrea gigas | CaCO₃ | Chitinase (Clp 1)) | Q1RQ16/EC 3.2.1.14 | |
| | (Calcite) | Chit 1-3 prot. | Q1RQ18-20/EC | |
| | | | 3.2.1.14 | |
| | | Ca-dependent protein | Q5Y1E3/EC 2.7.11.— | Protein phosphorylating |
| | | kinase | | |
| | | Matrix | Q86GD7/EC 3.4.24.— | Endopeptidase |
| | | metalloproteinase | | |
| A. rigida | CaCO₃ | Chitin synthase | Q288C6/EC 2.4.1.16 | |
| | (Arag + Calcite) | Asp.-rich proteins | Q5Y821-Q5Y830 | Asp-rich, Poly-Asp |
| P. yessoensis | CaCO₃ | Ca-ATPase | O96039/EC 3.6.3.8 | Ca transport |
| | (Calcite) | Nacrein-like | A0ZSF4/A0ZSF5/EC | Presumably carbonic anhydrase |
| | | proteins 1-2 | 4.2.1.1? | activity |
| | | SP-S | Q6BC45 | Acidic, Asp-Ser-rich, poly-Ser |
| | | MSP-1 | Q95YF6 | Asp-Ser-Gly-rich, repeats |
| Chordata | | | | |
| (Vertebrates) | | | | |
| Mus musculus | | GGPP synthase | Q9WTN0/EC 2.5.1.— | |
| | | Glycerophosphate | Q9ESM6/EC 3.1.—.— | Presumably glycerophosphoinositol |
| | | phosphodiesterase | | phosphodiesterase activity |
| | | Caspase 3 | P70677/EC 3.4.22.56 | |
| | | Phospholipase D1 | Q9Z280/EC 3.1.4.4 | |
| | | 11b-HSD1 (OH-steroid | P50172/EC 1.1.1.146 | |
| | | dehydrogenase) | | |
| | | Histone deacetylase 1 | O09106 | |
| | | (HDAC1) | | |
| | | Rho kinase | EC 2.7.11.14 | |
| | | Protein kinase C | EC 2.7.11.1 | |
| | | (PKC) | | |
| | | Janus kinases | P52332/Q62120/EC | |
| | | JAK1-3) | 2.7.10.2 | |
| | | E3 ubiquitin ligase Smurf1 | Q9CUN6/EC 6.3.2.— | |
| | | Vitamin D hydroxylase | EC 1.14.13.—, EC | |
| | | | 1.14.14.— | |
| | | P38 mitogen-activated | EC 2.7.11.24 | |
| | | protein kinase | | |
| | | Lipoprotein lipase | P11152/EC 3.1.1.34 | |
| | | NAD (P)H:quinone | Q64669/EC 1.6.5.2./EC | |
| | | oxidoreductase 1 | 1.6.99.2 | |
| | | Aromatases | P28649/EC 1.14.14.1 | |
| | | No synthase (endothelial | P70313/EC 1.14.13.39 | |
| | | isoform) | | |
| | | Thioredoxin peroxidase 2 | P35700/EC 1.11.1.15 | |
| | | (OSF-3) | | |
| | | Calcium ATPase | Q8R429/O55143/Q64518/ | |
| | | (SERCA 1-3) | EC 3.6.3.8 | |
| | | Phosphatidylinositol 3- | EC 2.7.1.1.#37/EC | |
| | | kinases I, | 2.7.1.153/EC | |
| | | II, III | 2.7.1.154 | |
| | | ERK/MAP kinase | EC 2.7.11.24 | |
| | | Lipoxygenase | EC 1.13.11.31 | |
| | | (Alox15) | | |
| | | Prostaglandin E2 | Q9R0Q7/EC 5.3.99.3 | |
| | | synthase | | |
| | | Cyclooxygenase-2 | Q05769/EC | |
| | | (COX-2) | 1.14.99.1 | |
| | | Tyrosine phosphatasese | P49446/Q60986/EC | |
| | | | 3.1.3.48 | |
| | | Phospholipase Cg Heme | Q62077/EC 3.1.4.11 | |
| | | oxygenase 1 | P14901/Q3TVV4/EC | |
| | | (HO-1) | 1.14.99.3 | |
| | | Lysosomal acid | Various enzymes | |
| | | hydrolases | (EC 3.—.—.—) | |
| | | Cysteine proteinases | P55097/EC | |
| | | (cathepsin K) | 3.4.22.38 | |
| | | H-ATPases | Various subunits/EC | |
| | | | 3.6.3.14 | |
| | | ADP-ribosyl cyclase | Q64277/P56528/EC | |
| | | (CD38) | 3.2.2.5 | |
| | | Adenyl cyclases 1 to 9 | EC 4.6.1.1 | |
| | | Superoxide dismutase | EC 1.15.1.1 | |
| | | Catalase | P24270/Q3TVZ1/Q3TXQ6/EC | |
| | | | 1.11.1.6 | |
| | | Glutathione peroxidase 1 | P11352/EC 1.11.1.9 | |
| | | (Gpx1) | | |
| | Bone | Tartrate- | Q05117/EC 3.1.3.2 | |
| | (HA) | resistant acid | | |
| | | phosphatase | | |
| | | Lactate dehydrogenase | P06151/P16125/P00342/EC | |
| | | | 1.1.1.27 | |
| | | MMPs (collagenases) | EC 3.4.24.— | |
| | | MMP-2/9 (gelatinase A-B) | P33434/P41245/EC | |
| | | | 3.4.24.24/35 | |
| | | Aggrecanases 1-2 | Q8BNJ2/Q9R001/EC | |
| | | | 3.4.24.— | |
| | | Ubiquitin-specific | Q8CBA4/Q99LG0/EC | |
| | | protease | 3.1.2.15 | |
| | | Calpain | EC 3.4.22.— | |
| | | Phospholipases A2 | Q9Z0Y2/EC 3.1.1.4 | |
| | | Carbonic anhydrase | P00920/EC 4.2.1.1 | |
| | | (II) | | |
| | | Procollagen C- | P98063/EC | |
| | | endopeptidase | 3.4.24.19 | |
| | | (BMP1) | | |
| | | Procollagen | EC 3.4.24.14 | |
| | | N-endopeptidase ADAMTS) | | |
| | | Prolyl-4 hydroxylases | Q60715/16/EC 1.14.11.2 | |
| | | Lysyl hydroxylases | Q9R0E1/E2/B9/EC | |
| | | 1-3 | 1.14.11.4 | |
| | | Galactosyl transferase | EC 2.4.1.— | |
| | | Lysyl oxidase 1 | Q08397/Q96JB6/EC | |
| | | and 4 | 1.4.3.— | |
| | | Protein-lysine 6- | P28300/EC 1.4.3.13 | |
| | | oxidase | | |
| | | Casein kinases II | Q60737/O54833/P67871/ | |
| | | | EC 2.7.11.1 | |
| | | Creatine kinases | EC 2.7.3.2 | |
| | | Tyrosine kinases | P97504/EC 2.7.10.2 | |
| | | Ser/Thr kinases | P36895/P36898/EC | |
| | | | 2.7.11.30 | |
| | | Alkaline phosphatases | P09242/EC 3.1.3.1 | |
| | | Protein phosphatases | EC 3.1.3.48/EC 3.1.3.16 | |
| | | Vitamin K-depend. g- | Q9QYC7/EC 6.4.—.— | |
| | | carboxylase | | |
| | | Tyrosylprotein | O60507/O60704/EC | |
| | | sulfotransferases 1-2 | 2.8.2.20 | |
| | | Transglutaminases 2 and | P21981/EC 2.3.2.13 | |
| | | factor XIIIA | | |
| | | Lipoprotein lipase | P11152/EC 3.1.1.34 | |
| | | Chondroitin sulf. | Q6ZQ11/Q6IQX7/EC | |
| | | synthase 1-2 | 2.4.1.175 | |
| | | Hyaluronan synthase 1-3 | Q61647/P70312/O08650/EC | |
| | | | 2.4.1.212 | |
| | | Chondroitinase | Q571E4/EC 3.1.6.4 | |
| | | Sialyl transferases | EC 2.4.99.— | |
| | | Carbohydrate | EC 2.8.2.— | |
| | | sulfotransferases | | |
| | | Osteocalcin | P04641 | Gla residues (g-carboxyGlu) |
| | | Osteopontin (BSP-1) | P10923 | Various P-Ser + O and N-linked |
| | | | | saccharide, RGD |
| | | BSP-2 | Q61711 | E-rich domain, poly-Glu, P-Ser, N- |
| | | | | linked sacch., RGD |
| | | SPARC/Osteonectin | P07214 | Follistatin-like, Kazal-like, EF- |
| | | | | hand, D-E-rich, N-linked sacch. |
| | | Tetranectin | P43025 | C-type lectin |
| | | Vitronectin | P29788/Q91X32 | Heparin-binding, RGD, S-Tyr, P-Ser, |
| | | | | N-linked sacch. |
| | | Fibronectins | P11276 and others | Fibronectin domains, RGDs, S-Tyr, |
| | | | | P-Ser, N-linked sacch. |
| | | Thrombospondins | P35441 and others | Heparin-binding, 3 EGF-like, some |
| | | | | TSP, vWFactor |
| | | Biglycan (SLRP) | P28653 | 12 L-rich repeats, 4 O and N-linked |
| | | | | saccharide |
| | | Decorin (SLRP) | P28654 | 12 L-rich repeats, 5 O and N-linked |
| | | | | saccharide |
| | | Osteoadherin, osteomodulin | O35103 | 12 L-rich repeats, D-E-rich, S-Tyr, |
| | | (SLRP) | | 6 N-linked saccharide |
| | | Osteoglycin, mimecan | Q62000 | 7 L-rich repeats, 1 N-linked |
| | | (SLRP) | | saccharide |
| | | Fibromodulin | P50608 | 12 L-rich repeats, poly-P, 5 N- |
| | | (SLRP) | | linked saccharide |
| | | Versican | Q62059 | Ig-like, 2 EGF-like, C-type lectin, |
| | | | | sushi, 17 N-linked sacch. |
| | | Aggrecan | Q61282 | Ig-like, C-type lectin, sushi, RGD, |
| | | | | 9 O and N-linked sacch. |
| | | Perlecan | A2BG65/Q05793 | LDL receptors, Ig-like, laminin |
| | | | | EGF-like, O and N-linked sacch. |
| | | Fetuins A-B | P29699/Q9QXC1 | Cystatin, P-Ser, N-linked |
| | | | | saccharide |
| Homo sapiens | | g- | EC 2.3.2.13 | |
| | | glutamyltransferase | | |
| | | (transglutaminase) | | |
| | | Ornithine decarboxylase | P11926/EC 4.1.1.17 | |
| | | (ODC) | | |
| | | Prostaglandin (PG) | Q16647/EC 5.3.99.4 | |
| | | I2 synthase | | |
| | | S- | P17707/EC 4.1.1.50 | |
| | | adenosylmethionine | | |
| | | decarboxylase | | |
| | | PACE4 convertase | P29122/EC 3.4.21.— | Endoprotease at R-X-X-R sites |
| | | (SPC4) | | |
| | E | MMP20 (enamelysin) | O60882/EC 3.4.24.— | Peptide hydrolase |
| | | Kallikrein-4 | Q9Y5K2/EC 3.4.21.— | Peptide hydrolase |
| | N | Alkaline phosphatase (AP- | P05186/EC 3.1.3.1 | |
| | | TNAP) | | |
| | | Phospholipase C | EC 3.1.4.11 | |
| | A | Calcium ATPase | EC 3.6.3.8 | |
| | | (SERCA2b) | | |
| | | ATP synthase Fl-b | P24539/EC 3.6.3.14 | |
| | | subunit | | |
| | M | Carbonic anhydrases | P00918/P23280/EC | |
| | | II, VI | 4.2.1.1 | |
| | | Amelogenin | Q99217/Q99218 | P-Ser, Pro-Gln-rich |
| | E | Enamelin | Q9NRM1/Q17RI5/Q8IWP4 | P-Thr, 10 N-linked saccharide |
| | | Enamelin-like prot. | Q96NF5 | Glu-Leu-rich |
| | L | Ameloblastin | Q9NP70/Q3B861/Q3B862 | 2 repeats |
| | | Amelotin | Q0P506 | Leu-Pro-Thr-rich |
| | (HA) | Biglycan | P21810 | 2 GAG |
| | | Tuftelin | Q9NNX1 | Glu-Leu-rich |
| | | TFP11 (Tuftelin | Q9UBB9 | Poly-Asp, P-Ser |
| | | interacting protein 11) | | |
| | | Calnexin | P27824 | Ca-binding, P-Ser, P-Thr, repeats |
| | | Fetuin-A (a-2-HS- | P02765 | Cystatins, P-Ser, O and N-linked |
| | | glycoprot.) | | saccharide |
| | | Cd63 (Lamp-3) | P08962/Q5TZP3 | N-linked saccharide |
| | | Annexin A2 (Anxa2) | P07355/Q8TBV2 | 4 repeats, P-Ser, P-Thr, P-Tyr |
| | | Lamp-1 | P11279 | |
| | D | DMP-1 (AG1) | Q13316/A1L4L3 | N-linked saccharide, RGD, |
| | | | | phosphorylated |
| | E | DPP, DSP, phosphophoryn | Q9NZW4 | N-linked saccharide, RGD, P-Ser, |
| | | | | Asp-Ser-rich, Ca-binding |
| | N | IBSP (integrin-binding | P21815 | Asp-Glu-rich, RGD, Poly-Glu, P-Ser, |
| | | sialoprotein, BSP II) | | N and O linked saccharide |
| | T | MEPE (matrix extracell. | Q9NQ76/A1A4X9 | RGD, N-linked saccharide, |
| | | phosphoglyc.) | | phosphorylated |
| | I | SPP1 (OPN, osteopontin) | P10451/Q567T5 | RGD, P-Thr, various P-Ser, N and O- |
| | | | | linked saccharide |
| Anser anser anser | | Ansocalcin | P83300 | |
| Gallus gallus | | Ovocleidin-17 | Q9PRS8 | |
| Strongylocentrotus | | SM32 | NP_999803 | |
| purpuratus | | | | |

TABLE 3

| | | | NBCI | SEQ ID |
|---|---|---|---|---|
| Name | Organism | | number | No. |
| Ansocalcin | Goose | Anser anser anser | P83300 | 1 |
| Ovocleidin- | Chicken | Gallus gallus | Q9PRS8 | 2 |
| 17 (OC-17) | | | | DNA: 79 |
| | | | | DNA: 20 |
| Perlucin | Abalone | Haliotis laevigata | P82596 | 3 |
| | | | | DNA: 80 |
| SM32 | Sea | Strongylocentrotus | NP_999803 | 4 |
| | urchin | purpuratus | | |
| N16.1 or | Oyster | Pinctada fucata | Q9TVT2 | 5 |
| N16N | | | | DNA: 78 |
| (slightly | | | | DNA: 19 |
| modified) | | | | |
| Silicatein a | Porifera | Suberites | Q2MEV3 | 6 |
| | | domuncula | | |
| Silicatein b | Porifera | Suberites | Q50IU7 | 7 |
| | | domuncula | | |
| Nacrein | Oyster | Pinctada fucata | Q27908 | 8 |
| Lustrin A | Abalone | Haliotis rufescens | O44341 | 9 |
| Amelogenin | Human | Homo sapiens | Q99217/ | 10/11 |
| | | | Q99218 | |
| Enamelin | Human | Homo sapiens | Q9NRM1/ | 12/13 |
| | | | Q8IWP4 | |

TABLE 4

| Protein | Databank ID | Seq. ID |
|---|---|---|
| EcmB (extracellular | Dictybase | 18 |
| matrix protein ST310) | DDB_G0269132 | |
| Signal peptide from | | 16 |
| EcmB | | DNA: 14 |
| Signal peptide from | | 17 |
| EcmB with extension | | DNA: 15 |
| St15 | Dictybase | 22 |
| | DDB0229932 | |
| Signal peptide from | | 15 |
| St15 | | |

TABLE 5

| Name | Template | Primer direction/name | Sequence 5′-3′ | Notes |
|---|---|---|---|---|
| ecmB promoter | ecmBGal-vector/ | ME_Xho_PecmB_for1 | CCGCTCGAG GGCTCCAACCAATCGTCC | Overhang |
| | Dictyostelium | | Seq. ID No. 23 | |
| | genomic DNA | | | |
| | | ME_PecmB_rev1 | GATTGCAATTTTAATAAATAAATATTTGATTGG | |
| | | | Seq. ID No. 34 | |
| | | ME_PecmB_SPecmB_rev2 | ATTAAATATATTTTATTCAT | |
| | | | GATTGCAATTTTAATAAATA | |
| | | | Seq. ID No. 35 | |
| | | ME_OPecmB_SPecmB_for1 | ATTGCAATCATGAATAAAATATATTTAA | |
| | | | TATTAATTTTAT | |
| | | | Seq. ID No. 24 | |
| | | ME_PecmB_rev1 | CCAATCAAATATTT | |
| | | | ATTTATTAAAATTGCAATC | |
| | | | Seq. ID No. 36 | |
| | | ME_PecmB_OSPecmB_rev1 | TCAAATATTTATTTATTAAAATTGCAATC | Signal |
| | | | ATGAATAAAATA | peptide |
| | | | Seq. ID No. 37 | |
| | | | Sequence reverse complementary: | |
| | | | TAT TTT ATT CAT GAT TGC AAT TTT | |
| | | | AAT AAA TAA ATA TTT GA | |
| | | | Seq. ID No. 38 | |
| | Re-PCR with | ME_SGecmB_for1 | ATG AAT AAA ATA TAT TTA ATA TTA | |
| | primer | | ATT TTA TTC | |
| | with overhang | | Seq ID No. 41 | |
| | | ME_SGecmB_BGiII_rev1 | AGATCT GGCTAAAATTATACCAACAAAAG | |
| | | | Seq. ID No. 42 | |
| | | | Sequence reverse complementary | |
| | | | cttttgttggtat aattttagccAGATCT | |
| | | | Seq. ID No. 43 | |
| | | ME_PecmB_SPecmB_for2 | TATTTATTAAAAT TGCAATCATGAAT AAAA- | |
| | | | TATATTTAAT | |
| | | | Seq. ID No. 44 | |
| EcmB signal | PdD56 vector | ME_PecmB_ÜSPecmB-rev1 | ATTGCAATC | Signal |
| peptide | | | ATGAATAAAATATATTTAATATTAATTTTAT | peptide |
| ampli- | | | Seq. ID No. 45 | |
| fication | | | | |
| with overhang | | | | |
| | | ME_SPecmB_BglII-rev1 | CACTTTTGTTGGTATAATTTTAGGCAGATCTTCC | |
| | | | Seq. ID No. 46 | |
| | | | Sequence reverse complementary: | |
| | | | GGA AGA TCT GGC TAA AAT TAT ACC | |
| | | | AAC AAA AGT G | |
| | | | Seq. ID No. 47 | |
| St15 | Genomic DNA | ME_St15_for1 | AGCGTTTATACTAA ACTGATACAATATTGG | First primer |
| | from Dicty | | Seq. ID No. 48 | in order to |
| | | | | obtain St15 in |
| | | | | a nested PCR |
| | | ME_St15_rev1 | GATATGTTTAGAGGTCGTTTAGTTGAG | |
| | | | Seq. ID No. 49 | |
| | | | Sequence reverse complementary: | |
| | | | CTCAACTAAACGAC CTCTAAACATATC | |
| | | | Seq. ID No. 50 | |
| | | ME_ÜPecmB_St15_for2 | ATTTATTAAAATTG CAATCATGTTTAAA | Nested PCR |
| | | | AAATTACTTTTC | with overhang |
| | | | Seq. ID No. 51 | into the |
| | | | or | ecmB promoter |
| | | | TTG CAATCATGTTTAAA AAATTACTTTTC | |
| | | | Seq. ID No. 52 | |
| | | ME_St15_BGiII_rev2 | AGATCT | End of St15 |
| | | | ATATCTAATATCAGTGGCTGAGAATAC | gene without/ |
| | | | Seq. ID No. 53 | with stop |
| | | | Sequence reverse complementary | codon |
| | | | GTA TTC TCA GCC ACT GAT ATT AGA | |
| | | | TAT AGATCT | |
| | | | Seq. ID No. 54 | |
| | | | or | |
| | | | GTA TTC TCA GCC ACT GAT ATT AGA | |
| | | | TAT AGATCTTCC | |
| | | | Seq. ID No. 55 | |
| | | ME_PecmB_ÜSt15_rev1 | TCAAATATTTATTTATTAAAATTGCAATC | |
| | | | ATGTTTAAAAA | |
| | | | Seq. ID No. 56 | |
| | | ME_PecmB_St15_rev3 | GAAAAGTAATTTTTTAAACAT | |
| | | | GATTGCAATTTTAATAAAT | |
| | | | Seq. ID No. 57 | |
| | | ME_Ncol_St15_for2 | CATGCCATGGATGTTTAAAAAATTACTTTTC | |
| | | | Seq. ID No. 58 | |
| | PCR product | ME_PecmB_St15_rev3 | GAAAAGTAATTTTTTAAACAT | Nested PCR |
| | of first | | GATTGCAATTTTAATAAAT | with |
| | ecmB-promoter | | Seq. ID No. 59 | overhang |
| | PCR | | Sequence reverse complementary | in St15 |
| | | | ATTTATTAAAATTGCAATCA | |
| | | | TGTTTAAAAAATTACTTTTC | |
| | | | Seq. ID No. 60 | |
| | Plasmid with | ME_PecmB_Ncol_Koz_ | ATTCAT TTTTT CCATGGG | |
| | EcmB-promoter | SPecmB_rev1 | ATTGCAATTTTAATAAATAAATATTTGATTGG | |
| | | | Seq. ID No. 61 | |
| Perlucin | Vector with | ME_CACC_Per_for_1b | CACCGGATGTCCTTTGGGT TTTCAC | CACC overhang |
| | perlucin | | Seq. ID No. 62 | |
| | | ME_CACC_Per_for2 | CACCGGA TGT CCT TTG GGT TTT CAC | CACC overhang |
| | | | CAA CAT CG | |
| | | | Seq. ID No. 63 | |
| | | EW_Per_rev_1 | TCTTTGTTGC AGATTGGCGT GAAGC | |
| | | | Seq ID No. 32 | |
| | | | Sequence reverse complementary | |
| | | | GCT TCA CGC CAA TCT GCA ACA AAG A | |
| | | | Seq. ID No. 64 | |

1. A method of identifying biomineralizing systems, comprising:
a) introducing at least one recombinant polynucleotide into at least one host cell, said recombinant polynucleotide;
a1) encoding at least one protein and/or polypeptide, and
a2) being suitable for expressing the encoded protein and/or polypeptide in said host cell,
b) expressing the protein encoded by the recombinant polynucleotide; and
c) investigating a biomineralizing action of the expressed protein.

2. The method as claimed in claim 1, wherein the recombinant polynucleotide has at least one nucleotide sequence encoding an amino acid sequence for controlling localization and/or time of expression of the protein and/or polypeptide.

3. The method as claimed in claim 2, wherein the recombinant polynucleotide encodes a fusion protein comprising the protein and/or polypeptide and a signal sequence for controlling localization and/or time of expression of the protein and/or polypeptide.

4. The method as claimed in claim 1, wherein the first recombinant polynucleotide encodes a protein and/or polypeptide from table 2.

5. The method as claimed in claim 4, wherein the host cell is a cell from the class of slime molds (Eumycetozoa).

6. The method as claimed in claim 5, wherein the host cell is a cell of the genus Dictyostelium.

7. The method as claimed in claim 1, wherein investigating the biomineralizing action comprises contacting the host cell with a solution of at least one salt.

8. An organic-inorganic composite material obtained by a method of claim 1.

9. A recombinant nucleic acid, comprising a recombinant nucleic acid that encodes a fusion protein comprising at least one protein and/or polypeptide with biomineralizing action and at least one amino acid sequence for influencing secretion of said fusion protein, and includes a corresponding promoter.

10. The recombinant nucleic acid as claimed in claim 9, wherein the encoded protein and/or polypeptide with biomineralizing action has a sequence which is at least 80% homologous to that of a protein and/or polypeptide from table 2.

11. A recombinant protein and/or polypeptide, comprising a fusion protein of at least one protein and/or polypeptide with biomineralizing action and an amino acid sequence for influencing secretion of said fusion protein, and said protein and/or polypeptide with biomineralizing action has a sequence which is at least 80% homologous to that of a protein and/or polypeptide from table 2.

12. A host cell, comprising a recombinant nucleic acid as claimed in claim 9.

13. The method as claimed in claim 1, wherein the first recombinant polynucleotide encodes a protein and/or polypeptide from table 3.

14. The method as claimed in claim 5, wherein the host cell is a cell of the species Dictyostelium discoideum.


SEQUENCE LISTING (83 sequences)

SEQ ID NO: 1 - protein, 132 aa, Anser anser anser
  SOURCE (1)..(132): /mol_type=“protein” /organism=“Anser anser anser”
NKCPKGWLDFRGSCYGYFGQELTWRKAEAWCKVIHAGCHLASLHSPEEHAAVARFIAKFQRREEEDNVWIGLHHWNQARVWIDGSKKRYSAWDDDELPRGKYCTVLEGSSGFMSWEDNACSERNPFVCKYSA

SEQ ID NO: 2 - protein, 142 aa, Gallus gallus
  SOURCE (1)..(142): /mol_type=“protein” /organism=“Gallus gallus”
DPDGCGPGWVPTPGGCLGFFSRELSWSRAESFCRRWGPGSHLAAVRSAAELRLLAELLNASRGGDGSGEGADGRVWIGLHRPAGSRSWRWSDGTAPRFASWHRTAKARRGGRCAALRDEEAFTSWAARPCTERNAFVCKAAA

SEQ ID NO: 3 - protein, 155 aa, Haliotis laevigata
  SOURCE (1)..(155): /mol_type=“protein” /organism=“Haliotis laevigata”
GCPLGFHQNRRSCYWFSTIKSSFAEAAGYCRYLESHLAIISNKDEDSFIRGYATRLGEAFNYWLGASDLNIEGRWLWEGQRRMNYTNWSPGQPDNAGGIEHCLELRRDLGNYLWNDYQCQKPSHFICEKERIPYTNSLHANLQQRDSLHANLQQR

SEQ ID NO: 4 - protein, 289 aa, Strongylocentrotus purpuratus
  SOURCE (1)..(289): /mol_type=“protein” /organism=“Strongylocentrotus purpuratus”
MKGVLFIVASLIAFATGQDCPAYYVRSQSGQSCYRYFNIPLAYQWASEFCEMVTPCGNGPAVMGTLAAPKSPQENMEIYRLVASFSQDNQMEREVWLGWNSMNPFMWENGAPAYPHGFSAFDSGGQAGANGWPVNTRNPFGMPPGFAPVMRRELGTIPGRQGPNRRMIPASQGPVWQVAELTGPTHAFVCEVPAGQTIVGQQQPTNPNFPNQPNQPFGPNQPNNPNQPFGPNQPNNPNQPNQPFAPNQPTTPNRPNQPFTPNQPNNPNQPNTPNTPNRPNQPNQPRLFQ

SEQ ID NO: 5 - protein, 131 aa, Pinctada fucata
  SOURCE (1)..(131): /mol_type=“protein” /organism=“Pinctada fucata”
MKCTLRWTITALVLLGICHLARPAYHKKCGRYSYCWIPYDIERDRRDNGGKKYCFCRYAWSPWQCNEEERYEWLRCGMRFYSLCCYTDDDNGNGNGNGNGNGLNYLKSLYGGYGNGNGEFREEYIDERYDN

SEQ ID NO: 6 - protein, 330 aa, Suberites domuncula
  SOURCE (1)..(330): /mol_type=“protein” /organism=“Suberites domuncula”
MLVTVVVLGLLGFASAAQPKFEFVEEWQLWKSTHSKMYESQLMELERHLTWLSNKKYIEQHNVNSHIFGFTLAMNQFGDLSELEYADYLGQYRIEDKKSGNYSKTFQRDPLQDYPEAVDWRTKGAVTAVKDQGDCGASYAFSAMGALEGANALAKGNAVSLSEQNIIDCSIPYGNHGCHGGNMYDAFLYVIANEGVDQDSAYPFVGKQSSCNYNSKYKGTSMSGMVSIKSGSESDLQAAVSNVGPVSVAIDGANSAFRFYYSGVYDSSRCSSSSLNHAMVVTGYGSYNGKKYWLAKNSWGTNWGNSGYVMMARNKYNQCGIATDASYPTL

SEQ ID NO: 7 - protein, 383 aa, Suberites domuncula
  SOURCE (1)..(383): /mol_type=“protein” /organism=“Suberites domuncula”
MSALKLIVALCVVHTSLGIAESVGKSKTAGLSDDGNYTADTKSVRLTPVLEFEEDWKQWTTDHHKVFSDVRERVDKYAVWRANKEYIDQHNQNAQRLGYTLKMNKFGDLTTKEFIEGIHCVQDYQPTNASHLNKKHNTHAFVDYGDFVRGGAGEGVRGVGDMPETMDWRTSGVVTKVKDQLRCGSSYAFSAMASLEGINALSYGSLVTLSEQNIVDCSVTYGNHGCACGDVNRALLYVIENDGVDTWKGYPSGGDPYRSKQYSCKYERQYRGASARGIVSLASGDENTLLTAVANSGPVSVYVDATSTSFQFYSDGVLNVPYCSSSTLSHALVVIGYGKYSGQDYWLVKNSWGPNWGVRGYGKLARNKGNKCGIATAASFPTL

SEQ ID NO: 8 - protein, 447 aa, Pinctada fucata
  SOURCE (1)..(447): /mol_type=“protein” /organism=“Pinctada fucata”
MYLHLTALCVVIPLCYGASMFKHDHYMDNGVRYPNGDGICKQLNETKCDAGFSYDRSICEGPHYWHTISKCFIACGIGQRQSPINIVSYDAKFRQRLPKLKFKPHMEKLKTEVTNHQNRAPEFEPEDGENLYVKLNNLVDGHYKFHNLHVHNGRTRRKGSEHSVNGRFTPMEAHLVFHHDDQTHFEPTRTKLGGAFPGHNDFVVVGVFLEVGDDGFGDEPDDEECKHILKGHHPDNNENGNGDNGNNGYNGDNGNNGDNGNNSYNGDNGNNGVNGNNGYNGDNGNNGDNGNNGYNGDNGNNGDNGNNGENGNNGENGNNGENGHKHGCRVKKAKHLSRILECAYRNDKVREFKKVGEEEGLDVHLTPEMALPPLKYRHYYTYEGSLTTPPCTESVLWVVQKCHVQVSRRVLHALRNVEGYKDGTTLRKYGTRRPTQKNKVTVYKSFK

SEQ ID NO: 9 - protein, 1428 aa, Haliotis rufescens
  SOURCE (1)..(1428): /mol_type=“protein” /organism=“Haliotis rufescens”
MERFLWVLCIAAGFSVNYGLRRAPYPCEPGLNVNCTTGECRLVFSCSLRRCGVRPECVDRSPVPSINCTIGKPTIDTNLQEISCAPDGSCPATTGCVRGPPAKPGVCCFNPSSGPPGPPRPPGPPRPPGPPQPDPNLLDPCFPGKNVNCTSGECRLMADCQHQSCPALPYCVAPSPNVTVPCPIGKSAIDRNLREFSCLRNRDACPRSTGCVVGAQGSAAVCCYRPPLVPGPTPTDPNPLDPCFPGKNVNCTAGECRLVADCSRKGCPAGPTCVDPSPVPSLNCDIGKPALNSYGNEISCAGGGACPVNTVCVAHPSGAPAVCCFKPAGPTTPQPPTIPQPPTTPSSPTGDPCEPGVNVNCTAGTCRLVVDCRFPGCPAVPKCVDPSSKPSLNCSIGDPALNPNLQEISCVGGAACPRNTACFAAPSGSPAVCCYTSGPPRPEPPSPSPPTGDPCEPGVNVNCTAGTCRLVEDCRIRGCPAVPKCIDRDPPLPPPPDVCPVGTPVLGADLKQLYCGRGGRRCPWNTYCVIHPADRYAVCCFGSGPASAIAPTSAPGPVDPCEPGVNVNCTIGVCRLVANCDYWPCPARPTCVDHSPEPSLNCTIGDPALNGKLEEFSCVGGRLCPLNTACLAAPSGSPAVCCYRPPVAITPAPTTVPIPVSTAAPTSAPGPVDPCQPGVNVNCTKGECRLVAICKYWPCFALPTCVDPSPPPSVECPVGKPALDEKLEEFSCKDCPFNTVCYKGAVCCVPWSGNRPSGPAGPAGPAGPERPATSVPLDPCTPGLNVNCTSGVCRLVEDCRRPGCPAVPTCIDRDPPLPPPPDVCPVGTPVLGRDLKQLYCGRGGKRCPGNTYCVIHPADRYAVCCFGSGPGPQPPIPTPPPPTTYPCTPANINCTAGECRLVAYCNAVPCGRTTPTCVDPSPPPTRKCPVGKPVLTPRLTEFRCYPRVRLCPGDSFCLRGPGDEPGVCCWDNRLRPTQGSGSGSGSGSGSGSSSSSSSGSTSGSGSGSGSGSSSGSGSGSSSASGSGSSSGSSSGSSSGSSSGSSSGSGSGSGSGSSSASGSGSSSGSSSGSSSGSGSGSSSASGSGSSSGSGSGSSSGSGSGSGSGSSSGSGSGSSSGSSSVSNSWTGSGSSSGSGSGSSSWSGSGSSSGTGSGSSSWFGSGSSSGSGSDSSSGSSSASGSGSSSGSSSGSGSGSSLWFGSGSSSGTGSGSSSGSSSGSGSDSSSGSSSGSTSGSSSGSGSASGSGTGSGKGASYDTDADSGSDNRSPGYLPQDPCTPGLYINCTAGTCRLTAWCLYNFCPAVPTCVDSSPDASGECPVGLPALNYFNKEVSCRTNLQCPSNTYCKSPGICCYRGPIARPRSSRYLAKYLKQGRSGKRLQKPGSCPAVRPDWAGICVVRCFCDNDCRGNLKCCSNGCGRTCQKPCFV

SEQ ID NO: 10 - protein, 191 aa, Homo sapiens
  SOURCE (1)..(191): /mol_type=“protein” /organism=“Homo sapiens”
MGTWILFACLLGAAFAMPLPPHPGHPGYINFSYEVLTPLKWYQSIRPPYPSYGYEPMGGWLHHQIIPVLSQQHPPTHTLQPHHHIPVVPAQQPVIPQQPMMPVPGQHSMTPIQHHQPNLPPPAQQPYQPQPVQPQPHQPMQPQPPVHPMQPLPPQPPLPPMFPMQPLPPMLPDLTLEAWPSTDKTKREEVD

SEQ ID NO: 11 - protein, 206 aa, Homo sapiens
  SOURCE (1)..(206): /mol_type=“protein” /organism=“Homo sapiens”
MGTWILFACLVGAAFAMPLPPHPGHPGYINFSYENSHSQAINVDRIALVLTPLKWYQSMIRPPYSSYGYEPMGGWLHHQIIPVVSQQHPLTHTLQSHHHIPVVPAQQPRVRQQALMPVPGQQSMTPTQHHQPNLPLPAQQPFQPQPVQPQPHQPMQPQPPVQPMQPLLPQPPLPPMFPLRPLPPILPDLHLEAWPATDKTKQEEVD

SEQ ID NO: 12 - protein, 1142 aa, Homo sapiens
  SOURCE (1)..(1142): /mol_type=“protein” /organism=“Homo sapiens”
MLVLRCRLGTSFPKLDNLVPKGKMKILLVFLGLLGNSVAMPMHMPRMPGFSSKSEEMMRYNQFNFMNGPHMAHLGPFFGNGLPQQFPQYQMPMWPQPPPNTWHPRKSSAPKRHNKTDQTQETQKPNQTQSKKPPQKRPLKQPSHNQPQPEEEAQPPQAFPPFGNGLFPYQQPPWQIPQRLPPPGYGRPPISNEEGGNPYFGYFGYHGFGGRPPYYSEEMFEQDFEKPKEEDPPKAESPGTEPTANSTVTETNSTQPNPKGSQGGNDTSPTGNSTPGLNTGNNPPAQNGIGPLPAVNASGQGGPGSQIPWRPSQPNIRENHPYPNIRNFPSGRQWYFTGTVMGHRQNRPFYRNQQVQRGPRWNFFAWERKQVARPGNPVYHKAYPPTSRGNYPNYAGNPANLRRKPQGPNKHPVGTTVAPLGPKPGPVVRNEKIQNPKEKPLGPKEQIIVPTKNPTSPWRNSQQYEVNKSNYKLPHSEGYMPVPNFNSVDQHENSYYPRGDSRKVPNSDGQTQSQNLPKGIVLGSRRMPYESETNQSELKHSSYQPAVYPEEIPSPAKEHFPAGRNTWDHQEISPPFKEDPGRQEEHLPHPSHGSRGSVFYPEYNPYDPRENSPYLRGNTWDERDDSPNTMGQKESPLYPINTPDQKEIVPYNEEDPVDPTGDEVFPGQNRWGEELSFKGGPTVRHYEGEQYTSNQPKEYLPYSLDNPSKPREDFYYSEFYPWSPDENFPSYNTASTMPPPIESRGYYVNNAAGPEESTLFPSRNSWDHRIQAQGQRERRPYFNRNIWDQATHLQKAPARPPDQKGNQPYYSNTPAGLQKNPIWHEGENLNYGMQITRMNSPEREHSSFPNFIPPSYPSGQKEAHLFHLSQRGSCCAGSSTGPKDNPLALQDYTPSYGLAPGENQDTSPLYTDGSHTKQTRDIISPTSILPGQRNSSEKRESQNPFRDDVSTLRRNTPCSIKNQLGQKEIMPFPEASSLQSKNTPCLKNDLGGDGNNILEQVFEDNQLNERTVDLTPEQLVIGTPDEGSNPEGIQSQVQENESERQQQRPSNILHLPCFGSKLAKHHSSTTGTPSSDGRQSPFDGDSITPTENPNTLVELATEEQFKSINVDPLDADEHSPFEFLQRGTNVQDQVQDCLLLQA

SEQ ID NO: 13 - protein, 1142 aa, Homo sapiens [sequence omitted]

SEQ ID NO: 14 - DNA, 54 bp, Dictyostelium
  source (1)..(54): /mol_type=“DNA” /organism=“Dictyostelium”
atgaataaaatatatttaatattaattttattcacttttgttggtataatttta

SEQ ID NO: 15 - DNA, 54 bp, Dictyostelium discoideum
  source (1)..(54): /mol_type=“DNA” /organism=“Dictyostelium discoideum”
aataaaatatatttaatattaattttattcacttttgttggtataattttagcc

SEQ ID NO: 16 - protein, 17 aa, Dictyostelium discoideum
  SOURCE (1)..(17): /mol_type=“protein” /organism=“Dictyostelium discoideum”
NKIYLILILFTFVGIIL

SEQ ID NO: 17 - protein, 18 aa, Dictyostelium discoideum
  SOURCE (1)..(18): /mol_type=“protein” /organism=“Dictyostelium discoideum”
NKIYLILILFTFVGIILA

SEQ ID NO: 18 - protein, 1053 aa, Dictyostelium discoideum
  SOURCE (1)..(1053): /mol_type=“protein” /organism=“Dictyostelium discoideum”
MNKIYLILILFTFVGIILANVEKAEVSCTCDENCNDGNKCTLDKCNNGCCSNTPININDNDECTVDTCNPKTGISHTPVNCDDGNSCTADSCLCGKGCQHVPIACDDNNACTVDSCSNSTGCCHTPLSCDDNNPCTVDSCSNSTGCCHTPINVDDHNACTEDKCTQSGGVTHTPIACDDKNACTVDSCSNSTGCCHTPLSCDDNNACTVDSCSNSTGCVHTPINVDDHNACTEDKCTQSGGVTHTPIACDDKNACTADSCSNSTGCCHTPITCDDNNACTVDSCSNSTGCCHTPINVDDNNACTEDKCTQSGGVTHTPIACDDKNACTVDSCSNSTGCVHTPLACDDKNPCTVDSCSNSTGCCHTPINVDDNNACTEDKCTQSGGVTHTPINCDDNNKCTVDSCSNSTGCCHTPMSCDDNNPCTVDSCSNSTGCVHTPINVDDNNACTEDKCTQNGGVTHTPIACDDKNACTVDSCSNSTGCCHTPLKCDDNNACTVDSCSNSTGCVHTPINVDDNNACTEDKCTQSGGVTHTPISCDDKNPCTIDSCSNSTGCVHTPMSCDDRNPCTSDFCSWEKGCQHVALSCNDFNACTMDSCSNSTGCTHTPIACDDKNACTVDSCSNSTGCVHTPLTCDDNNPCTVDSCSNSTGCCHTPINVDDHNACTEDKCTQSGGVTHTPIACDDKNACTVDSCSNSTGCCHTPLSCDDNNACTVDSCSNSTGCVHTPINVDDNNACTEDKCTQNGGVTHTPIACDDKNACTVDSCSNSTGCCHTPLKCDDNNPCTVDSCSNSTGCVHTPMNVDDNNACTEDKCTQNGGVTHTPIRCDDLNSCTADSCSNSTGCVHTPINCDDNNKCTADSCSNSTGCCHTPISCDDNNPCTVDSCSNSTGCCHTPINVDDNNPCTEDKCTQSGGVTHTPIGCNDNNACTVDSCSNSTGCTHTPMKCDDNNPCTIDSCSNSTGCVHTPMNCDDGNFCTLDSCCSTGCTHTPIIIDDNNPCTVDSCCNSTGVVHTPVDCNDNNVLTCDYCSIKQGGKCIHVPMTQCKTFGKCGDDL

SEQ ID NO: 19 - DNA, 225 bp, artificial sequences
  Synthetic Construct
gcgtatcataaaaaatgcggccgctatagctattgctggattccgtatgatattgaacgcgatcgctatgataacggcgataaaaaatgctacgacgttccggactacgcttctttgggtggttctagcccaagctcagagctccaccgcggtggcggccgcatcttttacccatacgatgttcctgactatgcgggctatccctatgacgtcccggactatgca

SEQ ID NO: 20 - DNA, 396 bp, Gallus gallus
  source (1)..(396): /mol_type=“DNA” /organism=“Gallus gallus”
gatccggatggctgcggcccgggctgggtgccgaccccgggcggctgcctgggcttttttagccgcgaactgagctggagccgcgcggaaagcttttgccgccgctggggcccgggcagccatctggcggcggtgcgcagcgcggcggaactgcgcctgctggcggaactgctgaacgcgagccgcggcggcgatggcagcggcgaaggcgcggatggccgcgtgtggattggcctgcatcgcccggcgggcagccgcagctggcgctggagcgatggcaccgcgccgcgctttgcgagctggcatcgcaccgcgaaagcgcgccgcggcgcgtttaccagctgggcggcgcgcccgtgcaccgaacgcaacgcgtttgtgtgcaaagcggcggcg

SEQ ID NO: 21 - protein, 122 aa, Dictyostelium
  SOURCE (1)..(122): /mol_type=“protein” /organism=“Dictyostelium”
MFKKLLFFTLTFALFALAICDNSIKITQTLSSAWEGHSTWNVVIENIGDKPIFGATIVAESGLSLERPDNLWSLDSLGNNRFGLPSWITQNGGLKAFNGTHTFGYTNAQPTSAVFSATDIRY

SEQ ID NO: 22 - protein, 20 aa, Dictyostelium
  SOURCE (1)..(20): /mol_type=“protein” /organism=“Dictyostelium”
MFKKLLFFTLTFALFALAIC

SEQ ID NO: 23 - DNA, 27 bp, artificial sequences
  Synthetic Construct
ccgctcgagggctccaaccaatcgtcc

SEQ ID NO: 24 - DNA, 40 bp, artificial sequences
  Synthetic Construct
attgcaatcatgaataaaatatatttaatattaattttat

SEQ ID NO: 25 - DNA, 96 bp, artificial sequences
  Synthetic Construct
ccaatcaaatatttatttattaaaattgcaatcatgaataaaatatatttaatattaattttattcacttttgttggtataattttaagatcttcc

SEQ ID NO: 26 - DNA, 96 bp, artificial sequences
  Synthetic Construct
ggaagatcttaaaattataccaacaaaagtgaataaaattaatattaaatatattttattcatgattgcaattttaataaataaatatttgattgg

SEQ ID NO: 27 - protein, 32 aa, artificial sequences
  Synthetic Construct
PIKYLFIKIAIMNKIYLILILFTFVGIILRSS

SEQ ID NO: 28 - DNA, 99 bp, artificial sequences
  Synthetic Construct
ccaatcaaatatttatttattaaaattgcaatcatgaataaaatatatttaatattaattttattcacttttgttggtataattttagccagatcttcc

SEQ ID NO: 29 - DNA, 99 bp, artificial sequences
  Synthetic Construct
ggaagatctggctaaaattataccaacaaaagtgaataaaattaatattaaatatattttattcatgattgcaattttaataaataaatatttgattgg

SEQ ID NO: 30 - protein, 33 aa, artificial sequences
  Synthetic Construct
PIKYLFIKIAIMNKIYLILILFTFVGIILARSS

SEQ ID NO: 31 - DNA, 26 bp, artificial sequences
  Synthetic Construct
caccggatgtcctttgggttttcacc

SEQ ID NO: 32 - DNA, 25 bp, artificial sequences
  Synthetic Construct
tctttgttgcagattggcgtgaagc

SEQ ID NO: 33 - DNA, 9267 bp, artificial sequences
  Synthetic Construct
ctcgagactagagctagataaaaaaaatttttatttatttttatttattttgaattaaatagattacaaattaattaatcccatcaaatctttaaaaaaaaatggtttaaaaaaacttgggttggttaattattatttgaaaattttaaaacccaaattaaaaaaaaaaaatgggattcaaaaatttttttttttttttttttttttttttttttttttttttttttttcagattgcataaaaagattttttttttttttttttcttatttcttaaaacaaataaattaaattaaaaaataaaaatcagatctacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggttccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggttccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggttactagtagtggtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactacttttacgtatggtgttcaatgcttttcaagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaagtaatctagttaaataaataaattatttaataaataataaaaaaacaaattgttgtaataatctaatattttcttttttttttaattttttttttttaaatcttaataattattaagttattttaattttttttttttttttttttttttttttttttttttttctatcaaaaaaatcaaatatatttaaaaaatttattatttacagatacattttgaatggtgaagataaatatatgcattagatgtaaaacagccaaagagtatgaaaatcaaaaagataaagcttatcgatttcgaaaaagtaaatagcaattattacaaaattcaatccgaatctacccaaataaattccaatgaaattgccgatttaaaaaagtttattaaagaagaagtcaataaaacttcttccaaaattgatttctttttagtttcttcaacagatgccctttcaaatccagaaaattattctctcttagaagtaaagtgtattaattgtcattctttgtgtcaaggaaaaaatttatatatttcatgtacaagagatggatgtcaaaacaatatttgctataattgtttaggaataaacataaacatatataatgttgttattaattctaaactttgccctccatgtttcaatgattcggtaatcaacaagaagtgtgccatgtgtagtaagaacggaactaaatgtaatttgaaccaagaatgtaaacttcatctttgtgcacagtgttctaaaaagtgtctatacattctgagagtcaaaactaattaaataaaatataaacttaatttctaaataaactcatttaaaaatatttaaataatatgaatttataactgtaattattgtattaaaaaattatataattatttaatgttaaaaatgtattaaaataattataaaaaaatataacaaaaattttcgtaaaaataatttgtaaaaaagctattaaaaatattatgaaaaaaaaattaaaaaaattattaaattgtttttgtaattaagctattaaaataattataaaaaaaaaatttttaaaattttaaaaatattttttgtaaaaaagtattaaaataattatgaaaaaaaaattttctaaaaaattaaaaaaaaaattaaaatatattttatgttaaaaacgtattaaaataactattaaaaaaattatatttaaaaaagtattaacttttttttaggtgtggttgtggggtggggtttaatatattataataaaaaattattttttgttcatttattattttcattgtatataatgtactcaacaacgttattattttttctttttttttttattgtatcaaaatcttctgttcttcaaaatgatcagattgaagtaaaatattttcaacttcttattgttatgtatcaaaaagaaaactgtgttgaaaagtcaatgacaggcgccgtaatttatgatgaatgtaatattcatggaagagttgaaacaaatagtactcatgcgcttttttatgatgacattgaaacaaataattcaagatgtaacaattttcgtaatttaacaaacttaattaaacttaatgaatgtattaatgacgagtttggagagtctattctttataaagaatataatgaaactgatgatggttatttgtttagagtggaagacagctttgttgaaattacttctctttcaatggattgtacaaaaaatagtaaaacaattattgaaaaattcaacatttgttcaaaatttgaaaatgtatatcatattacaaacattacacaagagaaatccaatagatttacatgtacagatccattgtgccactattgtaagaatgaaaacattcaaaacaatcttgattttaaaacaacaaagtgtactccaaagtatggtgcatctgattctgaatttttatcaacaatttacaatccaaagctcgatggctcaaataacggtatggaaaagtcagtaactcaagaaaaaaacatttcaaataatttaaaaattaatatatatttaattttctttttaattatttttttaattaaataaagttttattattttttaagagtaattattgctcttttttcatttgaaacaccagaagctaaacgtaattgttgttgactgaaattttttattttttttggggtaataggatttccttttttatgaagattaatatctttgactcgtgaaacattctttttaacttttgttttttctgttggtttatcatttgttttttcactaatttcaataccatcttgacgttcattcataacttcatcttttttttttcctgtttctgtatcttcttctatttttttttctttatctttttctttatcttcttcttgttcttcctcttctttttcttcttctgatactgcaggtgtttcttcttcttcttcttccgatattgtcggtttttctacttcttcttcttgttcttcttcttcttcttcttcttcttcttcttcttcttcttcttcttcttcttccggtaatttattaattatatttctttttttatatgaattacgtttggtttgtgcagtaatttccttacatagagtgcagctttcaagaaaaatttcaatttcttcgtttgttgcataataaccactgtctttgatatgattaaacatttttgattttcttaaatgctttccttctttaatatgaaaattatcgaattctaattcattaagaacaataagctcccctaatttaaaaaattagttaaaataaattaaaatgaacatgtataaagatggattttaccattttttgaaattctaaataacttttcttcatctccaatctttttgactgaaaaacgatttttaattgaagttattgttctgtgagtgttttgaatcgcccatttctctaaatcagtttgagatagtgttttataatctgaattgttatacacaacttttgctctattaaccaaatatttaaagatttcatcatcaactgaatattttgactttacgattcttgtccaaaaaacaatttctactactatcattttttatttataaaataatttaaatacaaaaatgaatttttttttttttaaaaaaaaaaaaatttgaaaaaaaaaaaaaaaaaattttaaaaaaaaaaaaaaaaaaaaaaaaaaaaaaaaaatcaaataaaaagtaaaaaataaaaaccgaaaaacattcattgtaatttcaaatgtcgaggccggcagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcgggatccattttatttaatatactaaataataaaaaagttaaaaaatgatcattggataaattttttataattataaataaagataataattttttttttaacaaaactaaaaataaaaataataaaataattgttaaaataggttttttttttttttttttttttttaataaatggtatttattaatttatttgttgtgtgtgtttttttttttataatatttttttttttagcattgaattaagaagaaatcaaattgattctagttcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccgtccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagcttgaacatcttcaccatccattttggatcttttatattatatttatttattgattatttttttgaattaattaaaaaaaaaaaaaatttcattttataatctcagaaacctcaaaaaaaaaaaaataaaaaataaaaaatataaaaaaataaaaataaaatcccaattttaaagcgaaaaaccacccatggtttgaaaatttcaatcaatttcaaataactttacttaaaaaaaacccattttttatttaaaaaa

SEQ ID NO: 34 - DNA, 33 bp, artificial sequences
  Synthetic Construct
gattgcaattttaataaataaatatttgattgg

SEQ ID NO: 35 - DNA, 40 bp, artificial sequences
  Synthetic Construct
attaaatatattttattcatgattgcaattttaataaata

SEQ ID NO: 36 - DNA, 33 bp, artificial sequences
  Synthetic Construct
ccaatcaaatatttatttattaaaattgcaatc

SEQ ID NO: 37 - DNA, 41 bp, artificial sequences
  Synthetic Construct
tcaaatatttatttattaaaattgcaatcatgaataaaata

SEQ ID NO: 38 - DNA, 41 bp, artificial sequences
  Synthetic Construct
tattttattcatgattgcaattttaataaataaatatttga

SEQ ID NO: 39 - DNA, 41 bp, artificial sequences; identical to SEQ ID NO: 37
  Synthetic Construct

SEQ ID NO: 40 - DNA, 40 bp, artificial sequences
  Synthetic Construct
attttattcatgattgcaattttaataaataaatatttga

SEQ ID NO: 41 - DNA, 33 bp, artificial sequences
  Synthetic Construct
atgaataaaatatatttaatattaattttattc

SEQ ID NO: 42 - DNA, 29 bp, artificial sequences
  Synthetic Construct
agatctggctaaaattataccaacaaaag

SEQ ID NO: 43 - DNA, 29 bp, artificial sequences
  Synthetic Construct
cttttgttggtataattttagccagatct

SEQ ID NO: 44 - DNA, 40 bp, artificial sequences
  Synthetic Construct
tatttattaaaattgcaatcatgaataaaatatatttaat

SEQ ID NO: 45 - DNA, 40 bp, artificial sequences; identical to SEQ ID NO: 24
  Synthetic Construct

SEQ ID NO: 46 - DNA, 34 bp, artificial sequences
  Synthetic Construct
cacttttgttggtataattttaggcagatcttcc

SEQ ID NO: 47 - DNA, 34 bp, artificial sequences
  Synthetic Construct
ggaagatctggctaaaattataccaacaaaagtg

SEQ ID NO: 48 - DNA, 30 bp, artificial sequences
  Synthetic Construct
agcgtttatactaaactgatacaatattgg

SEQ ID NO: 49 - DNA, 27 bp, artificial sequences
  Synthetic Construct
gatatgtttagaggtcgtttagttgag

SEQ ID NO: 50 - DNA, 27 bp, artificial sequences
  Synthetic Construct
ctcaactaaacgacctctaaacatatc

SEQ ID NO: 51 - DNA, 40 bp, artificial sequences
  Synthetic Construct
atttattaaaattgcaatcatgtttaaaaaattacttttc

SEQ ID NO: 52 - DNA, 29 bp, artificial sequences
  Synthetic Construct
ttgcaatcatgtttaaaaaattacttttc

SEQ ID NO: 53 - DNA, 33 bp, artificial sequences
  Synthetic Construct
agatctatatctaatatcagtggctgagaatac

SEQ ID NO: 54 - DNA, 33 bp, artificial sequences
  Synthetic Construct
gtattctcagccactgatattagatatagatct

SEQ ID NO: 55 - DNA, 36 bp, artificial sequences
  Synthetic Construct
gtattctcagccactgatattagatatagatcttcc

SEQ ID NO: 56 - DNA, 40 bp, artificial sequences
  Synthetic Construct
tcaaatatttatttattaaaattgcaatcatgtttaaaaa

SEQ ID NO: 57 - DNA, 40 bp, artificial sequences
  Synthetic Construct
gaaaagtaattttttaaacatgattgcaattttaataaat

SEQ ID NO: 58 - DNA, 31 bp, artificial sequences
  Synthetic Construct
catgccatggatgtttaaaaaattacttttc

SEQ ID NO: 59 - DNA, 40 bp, artificial sequences; identical to SEQ ID NO: 57
  Synthetic Construct

SEQ ID NO: 60 - DNA, 40 bp, artificial sequences; identical to SEQ ID NO: 51
  Synthetic Construct

SEQ ID NO: 61 - DNA, 50 bp, artificial sequences
  Synthetic Construct
attcattttttccatgggattgcaattttaataaataaatatttgattgg

SEQ ID NO: 62 - DNA, 25 bp, artificial sequences
  Synthetic Construct
caccggatgtcctttgggttttcac

SEQ ID NO: 63 - DNA, 33 bp, artificial sequences
  Synthetic Construct
caccggatgtcctttgggttttcaccaacatcg

SEQ ID NO: 64 - DNA, 25 bp, artificial sequences
  Synthetic Construct
gcttcacgccaatctgcaacaaaga

SEQ ID NO: 65 - DNA, 23 bp, artificial sequences
  Synthetic Construct
caccgcataccacaagaaatgcg

SEQ ID NO: 66 - DNA, 26 bp, artificial sequences
  Synthetic Construct
gcactttttatccccgttgtcgtatc

SEQ ID NO: 67 - DNA, 23 bp, artificial sequences
  Synthetic Construct
caccgaccctgacggctgtggac

SEQ ID NO: 68 - DNA, 25 bp, artificial sequences
  Synthetic Construct
cgccgccgctttgcaaacgaaggcg

SEQ ID NO: 69 - DNA, 35 bp, artificial sequences
  Synthetic Construct
ctcgaggatctttatgataatgaagagtctagttc

SEQ ID NO: 70 - DNA, 50 bp, artificial sequences; identical to SEQ ID NO: 61
  Synthetic Construct

SEQ ID NO: 71 - DNA, 79 bp, artificial sequences
  Synthetic Construct
caatcccatggaaaaaatgaataaaatatatttaatattaattttattcacttttgttggtataattttagccagatct

SEQ ID NO: 72 - DNA, 24 bp, artificial sequences
  Synthetic Construct
agatctggctaaaattataccaac

SEQ ID NO: 73 - DNA, 24 bp, artificial sequences
  Synthetic Construct
ctcgagggctccaaccaatcgtcc

SEQ ID NO: 74 - DNA, 40 bp, artificial sequences; identical to SEQ ID NO: 44
  Synthetic Construct

SEQ ID NO: 75 - DNA, 400 bp, artificial sequences
  Synthetic Construct
ggatgtcctttgggttttcaccaacatcgtcgaagctgctattggttctcaaccatcaagtcctcatttgctgaagctgctggttactgtagatacctagagtcgcatttggcgatcatatccaacaaagacgaagatagcttcattagaggctatgcaacaaggcttggcgaagcaggcaacgagtggcttggtgcaagcgatctgaatatagagggaagatggttatgggagggacaacgaaggatgaactataccaattggagtccgggacaaccagataacgctgggggaattgaacattgcttggagcttagacgtgatcttgggaattacctctggaatgactaccagtgtcaaaagccatcacatttcatctgcgagaaggaaaggattcctt

SEQ ID NO: 76 - DNA, 400 bp, artificial sequences
  Synthetic Construct
aaggaatcctttccttctcgcagatgaaatgtgatggcttttgacactggtagtcattccagaggtaattcccaagatcacgtctaagctccaagcaatgttcaattcccccagcgttatctggttgtcccggactccaattggtatagttcatccttcgttgtccctcccataaccatcttccctctatattcagatcgcttgcaccaagccactcgttgcctgcttcgccaagccttgttgcatagcctctaatgaagctatcttcgtctttgttggatatgatcgccaaatgcgactctaggtatctacagtaaccagcagcttcagcaaatgaggacttgatggttgagaaccaatagcagcttcgacgatgttggtgaaaacccaaaggacatcc

SEQ ID NO: 77 - protein, 133 aa, artificial sequences
  Synthetic Construct
GCPLGFHQHRRSCYWFSTIKSSFAEAAGYCRYLESHLAIISNKDEDSFIRGYATRLGEAGNEWLGASDLNIEGRWLWEGQRRMNYTNWSPGQPDNAGGIEHCLELRRDLGNYLWNDYQCQKPSHFICEKERIP

SEQ ID NO: 78 - DNA, 90 bp, artificial sequences
  Synthetic Construct
gcataccacaagaaatgcggaagatatagctattgctggataccgtacgacatcgaaagggatagatacgacaacggggataaaaagtgc

SEQ ID NO: 79 - DNA, 426 bp, artificial sequences
  Synthetic Construct
gaccctgacggctgtggacctggatgggtcccaaccccgggtggctgcctcggcttctttagccgggaacttagctggagccgtgccgagagcttttgccgacgttggggacctggatcacatctggccgctgtccggagcgcggctgaattgagacttctcgccgaactgctcaacgcctcaagaggaggcgatggatctggggaaggggctgatgggagagtgtggattggcctgcatcgaccagcaggaagccgatcttggagatggagtgacgggacagctccgcgttttgcttcctggcaccgaactgcaaaggcacgaagaggaggcaggtgcgctgcactaagggacgaagaggcctttacaagctgggcggcaagaccgtgcacggaaaggaacgccttcgtttgcaaagcggcggcg

SEQ ID NO: 80 - DNA, 465 bp, artificial sequences
  Synthetic Construct
ggatgtcctttgggttttcaccaacatcgtcgaagctgctattggttctcaaccatcaagtcctcatttgctgaagctgctggttactgtagatacctagagtcgcatttggcgatcatatccaacaaagacgaagatagcttcattagaggctatgcaacaaggcttggcgaagcattcaactattggcttggtgcaagcgatctgaatatagagggaagatggttatgggagggacaacgaaggatgaactataccaattggagtccgggacaaccagataacgctgggggaattgaacattgcttggagcttagacgtgatcttgggaattacctctggaatgactaccagtgtcaaaagccatcacatttcatctgcgagaaggaaaggattccttatacgaattccctccacgcaaatctacagcaaagagattcgcttcacgccaatctgcaacaaaga

SEQ ID NO: 81 - DNA, 119 bp, artificial sequences
  Synthetic Construct
ccatggaaaaaatgaataaaatatatttaatattaattttattcacttttgttggtataattttagccagatctacaagtttgtacaaaaaagcaggctccgcggccgcccccttcacc

SEQ ID NO: 82 - DNA, 51 bp, artificial sequences
  Synthetic Construct
aagggtgggcgcgccgacccagctttcttgtacaaagtggttactagtagt

SEQ ID NO: 83 - DNA, 64 bp, artificial sequences
  Synthetic Construct
tctagactgggatccagaattcagatctcggtaccaagcttactgcaggatgatcccgggcaat